(19) United States
(12) Patent Application Publication
(10) Pub. No.: US 2023/0346352 A1
(43) Pub. Date: Nov. 2, 2023

(54) ULTRASOUND DIAGNOSTIC APPARATUS

(71) Applicant: KONICA MINOLTA, INC., Tokyo (JP)

(72) Inventor: Tetsuya TANIGUCHI, Tokyo (JP)

(73) Assignee: KONICA MINOLTA, INC., Tokyo (JP)

(21) Appl. No.: 18/122,642

(22) Filed: Mar. 16, 2023

(30) Foreign Application Priority Data
Mar. 31, 2022 (JP) 2022-057920

(51) Int. Cl. A61B 8/08

(52) U.S. Cl. CPC A61B 8/5207

(57) ABSTRACT

An ultrasound diagnostic apparatus including: a transmitter; a hardware processor; a receiver; and an image generator. A first drive signal includes a third drive signal and a fourth drive signal. A second drive signal includes a fifth drive signal that is offset when the fifth drive signal and the third drive signal are added, and a sixth drive signal that generates an offset residual when the sixth drive signal and the fourth drive signal are added. The hardware processor causes the transmitter to provide a time delay to the first drive signal and the second drive signal so that the transmission ultrasound focuses to a same focal point, output the third drive signal and the fifth drive signal to the first transducer group, and output the fourth drive signal and the sixth drive signal to the second transducer group.

### REFERENCE TO RELATED APPLICATIONS

The entire disclosure of Japanese Patent Application No. 2022-057920 filed on Mar. 31, 2022, including description, claims, drawings and abstract is incorporated herein by reference in its entirety.

### TECHNICAL FIELD

The present invention relates to an ultrasound diagnostic apparatus.

### DESCRIPTION OF THE RELATED ART

Ultrasound diagnosis a simple operation in which the ultrasound probe is applied to the body surface of the patient’s subject body or from the inside of the body cavity to obtain ultrasound images of the heart and fetus. The ultrasound diagnosis can perform tests repeatedly since it is safe. The ultrasound diagnostic apparatus used to perform such ultrasound diagnosis is known.

As a technique for displaying such ultrasound diagnostic images, tissue harmonic imaging (THI), which produces images with good contrast by imaging the higher harmonic wave components (components not included in the transmission ultrasound) generated in living tissue.

In addition, there is known an ultrasound diagnostic apparatus in which, of the plurality of transducers of the ultrasound probe, the first drive signal is output to the first transducer group located inside the transmission aperture, the second drive signal is output to the second transducer group located in the outer side than first transducer group, the frequency band of the frequency power spectrum of the transmitted pulse of the first drive signal is wider including the frequency band of the frequency power spectrum of the transmitted pulse of the second drive signal and includes a higher component than frequency band of the frequency power spectrum of the transmitted pulse of the second drive signal (see JP 2018-114195 A). The ultrasound diagnostic apparatus in JP 2018-114195 A suppresses acoustic noise and improves penetration (depth).

### SUMMARY OF THE INVENTION

However, THI requires high sound pressure (ultrasonic sound pressure) to generate higher harmonic waves. High sound pressure can be achieved by strong ultrasonic transmission and ultrasound wave collection. Here, FIG. 25 illustrates the intensity of ultrasonic sound pressure in the presence of the acoustic shield. FIG. 25 shows the sound pressure intensity of the transmission ultrasound of an ultrasound probe 2L.

FIG. 25 shows the intensity of the sound pressure of the transmission ultrasound when there is an acoustic shield AS1 on the probe side of the transmission focal point of the transmission ultrasound of the ultrasound probe 2L with linear scanning method. In FIG. 25, in the transmission ultrasound of the ultrasound probe 2L, the higher sound pressure is represented as white and the lower sound pressure as black. The acoustic shield AS1 is a highly reflective, highly attenuating tissue or other shields, represented by the shaded pattern area on the figure. The presence of the acoustic shield AS1 does not increase the sound pressure of the transmission ultrasound and reduces the amount of higher harmonic wave component generation. This results in a backward shadow (AS2) with low sound pressure, which extends to the deep portion.

In particular, small-diameter convex-type probes, as typified by transvaginal probes, are susceptible to this effect because there is a limit to aperture expansion in the azimuthal direction even if the number of elements used for transmission is increased.

The ultrasound diagnostic apparatus in JP 2018-114195 A, while useful for acoustic noise suppression and penetration improvement, images higher harmonic wave components that are highly sound pressure dependent and has weak shadow tolerance in the presence of the acoustic shield.

The problem of the present invention is to obtain good ultrasound images in the presence of the acoustic shield.

To achieve at least one of the abovementioned objects, according to an aspect of the present invention, the ultrasound diagnostic apparatus reflecting one aspect of the present invention is An ultrasound diagnostic apparatus that generates ultrasound image data based on a reception signal obtained by an ultrasound probe that transmits transmission ultrasound to a subject and receives reception ultrasound from the subject, wherein the ultrasound probe includes a transmission aperture including a plurality of transducers, and the transmission aperture includes a plurality of transducer groups including at least a first transducer group that is arranged in an inner side of the transmission aperture and a second transducer group that is arranged in an outer side than the first transducer group, the ultrasound diagnostic apparatus including: a transmitter that generates a drive signal and outputs the drive signal to the plurality of transducer groups; a hardware processor that causes the transmitter to output a plurality of drive signals including at least a first drive signal and a second drive signal to the plurality of transducer groups for one focusing line; a receiver that receives a plurality of reception signals corresponding to the plurality of drive signals from the ultrasound probe; and an image generator that calculates the plurality of reception signals and generates ultrasound image data, wherein the first drive signal includes a third drive signal and a fourth drive signal, the second drive signal includes a fifth drive signal that is offset when the fifth drive signal and the third drive signal are added, and a sixth drive signal that generates an offset residual when the sixth drive signal and the fourth drive signal are added, and the hardware processor causes the transmitter to provide a time delay to the first drive signal and the second drive signal so that the transmission ultrasound focuses to a same focal point, output the third drive signal and the fifth drive signal to the first transducer group, and output the fourth drive signal and the sixth drive signal to the second transducer group.

### BRIEF DESCRIPTION OF THE DRAWINGS

The advantages and features provided by one or more embodiments of the invention will become more fully understood from the detailed description given hereinafter and the appended drawings which are given by way of illustration only, and thus are not intended as a definition of the limits of the present invention, and wherein:

FIG. 1 is an external view of an ultrasound diagnostic apparatus in an embodiment of the present invention;

FIG. 2 is a block diagram showing the functional configuration of the ultrasound diagnostic apparatus;

FIG. 3 shows a block diagram of the transmitter’s functional configuration;

FIG. 4A shows the transmission intensity of the transmission ultrasound for the transducer channel of the ultrasound probe;

FIG. 4B shows the sound pressure rise contribution to the transmission center direction of the transducer channel of the ultrasound probe;

FIG. 5 shows the ultrasound beams corresponding to the first and second transducer groups output from the ultrasound probe;

FIG. 6A shows an example of the time characteristic of the signal intensity of the transmission ultrasound corresponding to the fourth drive signal;

FIG. 6B shows an example of the time characteristic of the signal intensity of the transmission ultrasound corresponding to the sixth drive signal;

FIG. 7A shows an example of the time characteristics of the signal intensities of the transmission ultrasound corresponding to the fourth drive signal of FIG. 6A and the transmission ultrasound corresponding to the sixth drive signal of FIG. 6B, and their combined signal;

FIG. 7B shows an example of the power spectra of the signal intensities of the transmission ultrasound corresponding to the fourth drive signal of FIG. 6A and the transmission ultrasound corresponding to the sixth drive signal of FIG. 6B, and their combined signal, and the transmission band of the ultrasound probe;

FIG. 8 shows the preferred power spectrum of the signal intensity of the transmission ultrasound of the first and second transducer groups;

FIG. 9A shows the time characteristics of the signal intensity of the drive signal of the first drive waveform;

FIG. 9B shows the time characteristics of the signal intensity of the transmission ultrasound corresponding to the drive signal of the first drive waveform;

FIG. 10A shows the time characteristics of the signal intensity of the drive signal of the second drive waveform;

FIG. 10B shows the time characteristics of the signal intensity of the transmission ultrasound corresponding to the drive signal of the second drive waveform;

FIG. 11A shows the time characteristics of the signal intensity of the drive signal of the third drive waveform;

FIG. 11B shows the time characteristics of the signal intensity of the transmission ultrasound corresponding to the drive signal of the third drive waveform;

FIG. 12A shows the time characteristics of the signal intensity of the drive signal of the fourth drive waveform;

FIG. 12B shows the time characteristics of the signal intensity of the transmission ultrasound corresponding to the drive signal of the fourth drive waveform;

FIG. 13A shows the time characteristics of the signal intensity of the drive signal of the fifth drive waveform;

FIG. 13B shows the time characteristics of the signal intensity of the transmission ultrasound corresponding to the drive signal of the fifth drive waveform;

FIG. 14A shows the time characteristics of the signal intensity of the drive signal of the sixth drive waveform;

FIG. 14B shows the time characteristics of the signal intensity of the transmission ultrasound corresponding to the drive signal of the sixth drive waveform;

FIG. 15A shows the time characteristics of the signal intensities of the transmission ultrasound corresponding to the drive signals of the first and second drive waveforms;

FIG. 15B shows the power spectra of the signal intensities of the transmission ultrasound corresponding to the drive signals of the first and second drive waveforms;

FIG. 16A shows the time characteristics of the signal intensity of the transmission ultrasound corresponding to the drive signals of the third and fourth drive waveforms;

FIG. 16B shows the time characteristic of the first combined waveform of the transmission ultrasound corresponding to the drive signals of the third and fourth drive waveforms;

FIG. 17 shows the power spectra of the signal intensities of the transmission ultrasound corresponding to the drive signals of the third and fourth drive waveforms and the combined signal of the first combined waveform;

FIG. 18 shows the time characteristics of the signal intensity of the transmission ultrasound corresponding to the drive signals of the fifth and fourth drive waveforms;

FIG. 19A shows the time characteristics of the signal intensity of the transmission ultrasound corresponding to the drive signals of the sixth and fourth drive waveforms;

FIG. 19B shows the time characteristics of the second combined waveform of the transmission ultrasound corresponding to the drive signals of the sixth and fourth drive waveforms;

FIG. 20 shows the power spectra of the signal intensities of the transmission ultrasound corresponding to the drive signals of the sixth and fourth drive waveforms and the combined signal of the second combined waveform;

FIG. 21A shows an ultrasound image of the first subject in the fourth comparative example;

FIG. 21B shows an ultrasound image of the first subject in the first example;

FIG. 22A is a 3D graph of the brightness values of the partial area of FIG. 21A;

FIG. 22B is a 3D graph of the brightness values of the partial area of FIG. 21B;

FIG. 23A shows an ultrasound image of the second subject in the fourth comparative example;

FIG. 23B shows an ultrasound image of the second subject in the first example;

FIG. 24A is a 3D graph of the brightness values of the partial area of FIG. 23A;

FIG. 24B is a 3D graph of the brightness values of the partial area of FIG. 23B; and

FIG. 25 shows the sound pressure intensity of the transmission ultrasound of the ultrasound probe.

### DETAILED DESCRIPTION

Referring to the accompanying drawings, the present embodiment according to the present invention is described in detail. However, the scope of the invention is not limited to the disclosed embodiments or illustrated examples.

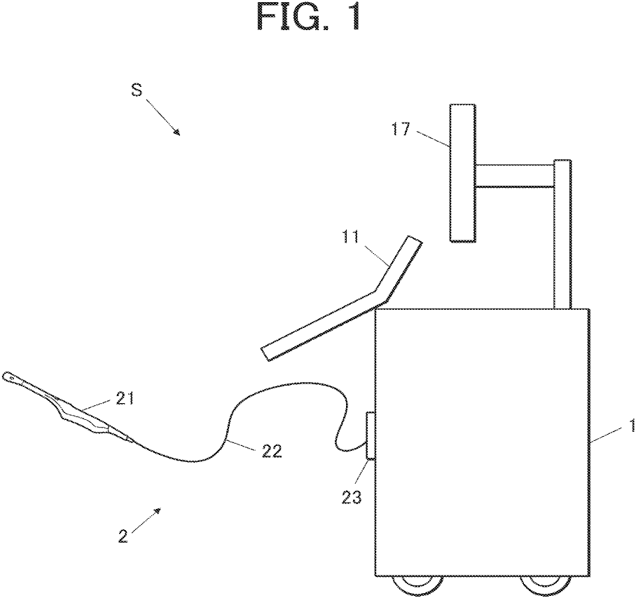

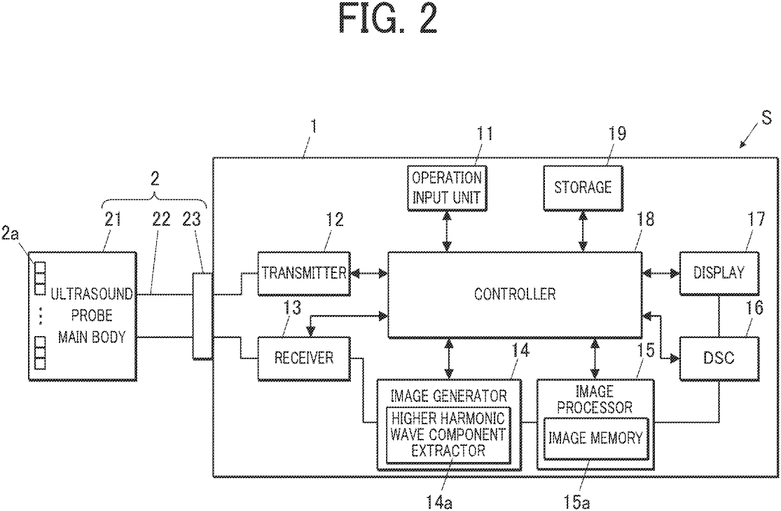

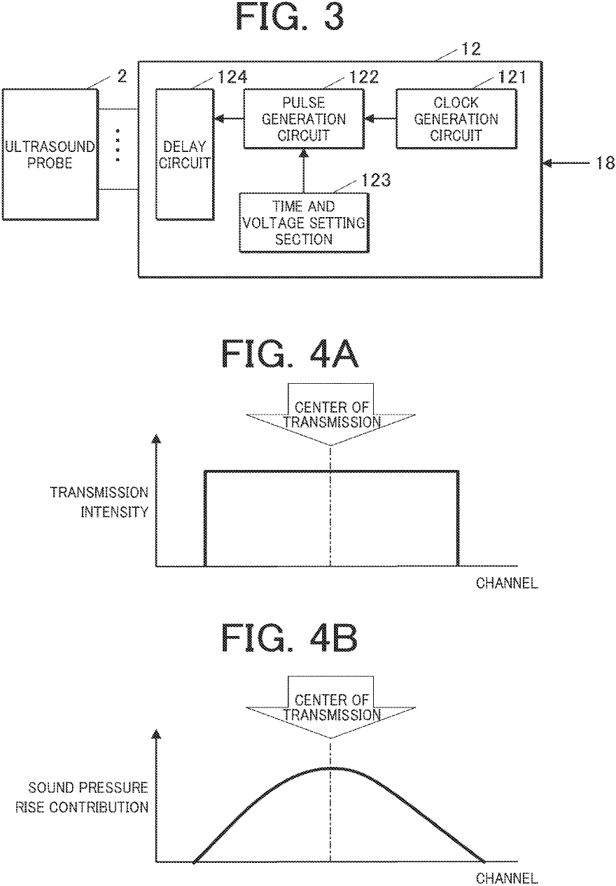

First, the apparatus configuration of the ultrasound diagnostic apparatus S of the present embodiment is described with reference to FIG. 1 to FIG. 3. FIG. 1 is an external diagram of the ultrasound diagnostic apparatus S according to the present embodiment. FIG. 2 is a block diagram showing a functional configuration of the ultrasound diagnostic apparatus S. FIG. 3 is a block diagram showing a functional configuration of a transmitter 12.

As shown in FIG. 1 and FIG. 2, the ultrasound diagnostic apparatus S according to the present embodiment includes an ultrasound diagnostic apparatus main body 1 and an ultrasound probe 2. The ultrasound probe 2 transmits the ultrasound (the transmission ultrasound) to the subject such as a live body (not shown) and receives reception ultrasound including reflected ultrasound reflected on the subject and scattered ultrasound. The ultrasound diagnostic apparatus main body 1 is connected to the ultrasound probe 2. The ultrasound diagnostic apparatus main body 1 transmits a drive signal of an electric signal to the ultrasound probe 2 and allows the ultrasound probe 2 to transmit the transmission ultrasound to the subject. The ultrasound diagnostic apparatus main body 1 images the internal state of the subject as the ultrasound image data based on the reception signal which is an electric signal generated in the ultrasound probe 2 in response to the reception ultrasound from the subject received by the ultrasound probe 2.

The ultrasound probe 2 has an ultrasound probe main body 21, a cable 22, and a connector 23. The ultrasound probe main body 21 is the header of the ultrasound probe 2, which transmits and receives ultrasound waves. The cable 22 is the cable that is connected between the ultrasound probe main body 21 and connector 23 and through which the drive signal for the ultrasound probe main body 21 and the ultrasound the reception signal flow. The connector 23 is a plug connector for connection to the receptacle connector (not shown) on the ultrasound diagnostic apparatus main body 1.

The ultrasound diagnostic apparatus main body 1 is connected to the ultrasound probe main body 21 via connector 23 and the cable 22. The ultrasound probe main body 21, which includes transducers 2a made of piezoelectric elements and an acoustic lens that focuses the transmission ultrasound toward the focal point, is the main body part of a body cavity probe (transvaginal probe) for examining the subject’s body cavity (for example, in the vagina). The plurality of transducers 2a are arranged in a semicircle of 180 degrees, for example. The present embodiment uses, for example, the ultrasound probe 2 with 192 transducers 2a. The transducers 2a may be arranged in a two-dimensional array. The number of transducers 2a can be set arbitrarily. In the present embodiment, the ultrasound probe 2 is an electronic scan body cavity probe, but any of electronic or mechanical scanning method can be used, and any linear, sectoral, or convex scanning method can also be used.

The frequency bandwidth of the ultrasound probe 2 is preferably 100% or greater of the -20 dB fractional bandwidth of the transmit/receive sensitivity, and more preferably 120%or greater. Specifically, if the central frequency of the ultrasound probe 2 is 6 MHz, the -20 dB bandwidth is preferably wider than 3 to 9 MHz and more preferably 2.4 to 9.8 MHz. By using a wideband ultrasound probe 2, it is possible to transmit both high frequencies, which are the drive force for shallow portion higher harmonic wave generation, and low frequencies, which are important for ensuring penetration, and to obtain good signal-to-noise ratio (S/N) over shallow to deep portions. Furthermore, since the upper bandwidth limit of the offset residual component is limited by the bandwidth of the ultrasound probe, the use of a wideband ultrasound probe 2 contributes to improved resolution.

As shown in FIG. 2, for example, the ultrasound diagnostic apparatus main body 1 includes an operation input unit 11, a transmitter 12, a receiver 13, an image generator 14, an image processor 15, a DSC (digital scan converter) 16, a display 17, a controller 18 (hardware processor), and a storage 19.

For example, the operation input unit 11 includes various switches, buttons, a track ball, a mouse, a keyboard, etc. to input a command to instruct the start of diagnosis or input of data such as individual information of the subject, receives the operation input from the user such as a physician and a technician and outputs the operation signal to the controller 18.

The transmitter 12 is a circuit which supplies an electric drive signal to the ultrasound probe 2, and allows the ultrasound probe 2 to generate the transmission ultrasound according to control by the controller 18. The transmitter 12 also divides the plurality of transducers 2a of the transmission aperture of the ultrasound probe 2 into two groups: a first transducer group located in an inner side of the transmission aperture (in the center of the transmission aperture) and a second transducer group located in the outer side than first transducer group which is located in the inner side (at the edge portion of the transmission aperture). The transmitter 12 generates drive signals with different drive waveforms for the same focal point, provides time delays to plurality of drive signals so that the transmission ultrasound focus to the same focal point, and outputs them to the first transducer group and the second transducer group. In the embodiment, “different drive waveforms” refers to different drive control signals. The transmission aperture is an aperture of a fixed number (for example, 46) of a series of transducer groups that output drive signals for ultrasound transmission among all the transducers 2a of the ultrasound probe 2.

For example, as shown in FIG. 3, the transmitter 12 includes, a clock generation circuit 121, a pulse generation circuit 122, a time and voltage setting section 123, and a delay circuit 124.

The clock generation circuit 121 is a circuit which generates a clock signal which determines the transmission timing and the transmission frequency of the drive signal. The pulse generation circuit 122 is a circuit which generates a pulse signal as a drive signal at a predetermined cycle. For example, the pulse generation circuit 122 switches the voltage of 3 values (+HV/0 (GND)/-HV), and outputs the above to generate the drive signal by a rectangular wave. Here, the amplitude of the pulse signal is the same at the positive polarity and the negative polarity but the present invention is not limited to the above. According to the present embodiment, the voltage of 3 values is switched to output the drive signal, but the present invention is not limited to 3 values, and a suitable number of values such as 5 values (+HV/+MV/0(GND)/-MV/-HV) can be set. Preferably, the number is 5 values or less. With this, the freedom of control of the frequency components can be enhanced at a low cost, and the transmission ultrasound with high resolution can be obtained.

The time and voltage setting section 123 sets for each section the continuing time that the drive signal output from the pulse generation circuit 122 is the same voltage level and the voltage level. That is, the pulse generation circuit 122 outputs the drive signal with the pulse waveform according to the continuing time of each section and the voltage level set by the time and voltage setting section 123. The continuing time and the voltage level of each section set by the time and voltage setting section 123 can be varied by input operation by the operation input unit 11, for example.

The delay circuit 124 is a circuit which sets the delay time of the transmission timing of the drive signal for each individual path corresponding to each transducer and delays the transmission of the drive signal for the set delay time and focuses the transmission beam including the transmission ultrasound.

According to the control by the controller 18, the transmitter 12 sequentially switches the transducers 2a of the transmission aperture to which the drive signal is supplied shifting a predetermined number for each transmission/reception of the ultrasound. The scanning is performed by supplying the drive signal to the transducers 2a selected to output.

According to the above-described embodiment, the pulse inversion method can be performed to extract the higher harmonic wave component of THI. The pulse inversion method offsets the fundamental wave component and extracts only the higher harmonic wave component by adding the reception signals (echo signals) at the time of transmission of two drive signals whose fundamental waves are inverted in phase with each other. In other words, when the pulse inversion method is performed, the transmitter 12 is able to transmit, as (transmission) drive signals, a first drive signal of first drive waveform and a second drive signal of second drive waveform having the signal intensity (voltage) with the polarity inverted from the first drive signal on the same scanning line with a time interval in between. The first and second drive signals with inverted polarity are the symmetric pulse inversion (hereinafter referred to as symmetric PI) drive signals in the present invention.

The transmitter 12 can also transmit a second drive signal that is polarity-reversed with at least one of the plurality of duties of the first drive signal being different. When the drive signals corresponding to the first and second drive signals are added, there is a component that remains to be added, and this component is the offset residual. The first and second drive signals with offset residuals are the asymmetric pulse inversion (hereafter referred to as asymmetric PI) drive signals in the present invention.

Thus, the symmetry/asymmetry of the PI in the present embodiment is defined by the positive/negative symmetry of the (transmission) drive signal and does not consider symmetry by other symmetry axes such as time-reversal symmetry. It also does not consider positive/negative asymmetry caused by drive device characteristics, such as asymmetry in the rise/fall time of the drive voltage or differences in overshoot/undershoot characteristics. In other words, the symmetry/asymmetry of the PI is defined by the positive/negative symmetry of the drive (control) signal.

Here, the first drive signal includes the third and fourth drive signals, and the second drive signal includes the fifth and sixth drive signals. The third and fifth drive signals are the drive signals in the symmetric PI relationship, and the fourth and sixth drive signals are the drive signals in the asymmetric PI relationship. The transmitter 12, configured as described above, generates a first drive signal including the third drive signal and the fourth drive signal, and a second drive signal of a second drive waveform including the fifth drive signal and the sixth drive signal. The transmitter 12 can output the first drive signal so that the third drive signal is output to the first transducer group located in the inner side of the transmission aperture and the fourth drive signal is output to the second transducer group located in the outer side of the transmission aperture, so that they are in the same focus, and then output the second drive signal so that the fifth drive signal is output to the first transducer group located in the inner side of the transmission aperture and the sixth drive signal is output to the second transducer group located in the outer side of the transmission aperture, so that they are in the same focus as that of the first drive signal. The relationship between the first through sixth drive signals and the first and second transducer groups is summarized in Table I below.

TABLE 1

DRIVE SIGNAL

| | | |
|---|---|---|
| VIBRATOR GROUP | FIRST TRANSMISSION | SECOND TRANSMISSION |
| | FIRST DRIVE SIGNAL | SECOND DRIVE SIGNAL |
| | \|\| | \|\| |
| FIRST VIBRATOR GROUP | THIRD DRIVE SIGNAL | FIFTH DRIVE SIGNAL |
| | + | + |
| SECOND VIBRATOR GROUP | FOURTH DRIVE SIGNAL | SIXTH DRIVE SIGNAL |

The receiver 13 is a circuit which receives an electric reception signal from the ultrasound probe 2 according to the control by the controller 18. The receiver 13 includes, for example, an amplifier, an A/D conversion circuit, and a phase adding circuit. The amplifier is a circuit which amplifies the reception signal for each individual path corresponding to each transducer 2a at a predetermined amplifying rate. The A/D conversion circuit is a circuit for analog-digital conversion (A/D conversion) of the amplified reception signal. The phase adding circuit is a circuit which applies delay time to the A/D converted the reception signal for each individual path corresponding to the transducer 2a to adjust the time phase, and adds the above (phase adding) to generate the focusing line data.

The image generator 14 performs an envelope curve detection process and logarithm amplifying on the focusing line data from the receiver 13 and performs gain adjustment to convert the brightness. With this, B-mode image data is generated. That is, the B-mode image data shows the intensity of the reception signal with brightness. The B-mode image data generated by the image generator 14 is transmitted to the image processor 15. The image generator 14 includes a higher harmonic wave component extractor 14a and generates the B-mode image data from the higher harmonic wave component extracted by the higher harmonic wave component extractor 14a.

The higher harmonic wave component extractor 14a performs the pulse inversion method and extracts the higher harmonic wave component from the focusing line data of the reception signal output from the receiver 13. More specifically, the higher harmonic wave component extractor 14a can extract the higher harmonic wave components by adding (combining) the focusing line data of the first the reception signal corresponding to the first drive signal and the focusing line data of the second the reception signal corresponding to the second drive signal, removing the fundamental wave component included in the reception signal, and then performing the filter process as necessary. The higher harmonic wave component of the odd order higher harmonic wave component can be obtained by subtracting (combining) the same first and second reception signal focusing line data, removing the even-order higher harmonic wave component, and then performing the filter process as necessary. The even-order higher harmonic wave component and the odd-order higher harmonic wave component extracted as described above can be added (combined) after performing the phase adjustment process with the all pass filter as necessary, and the higher harmonic wave of the even-order and the odd-order can be combined without canceling (offset) to obtain a broadband reception signal.

The image processor 15 includes an image memory 15a including a semiconductor memory such as a DRAM (Dynamic Random Access Memory). The image processor 15 stores, in the image memory 15a, B-mode image data output from the image generator 14 in the unit of frames. The image data in the unit of frames may be called the ultrasound image data or frame image data. The image processor 15 suitably reads the ultrasound image data stored in the image memory 15a and outputs the data to the DSC 16.

The DSC 16 performs a process such as a coordinate conversion on the ultrasound image data received by the image processor 15 to convert the data to image signals, and outputs the image signals to the display 17.

Various display apparatuses can be applied as the display 17, for example, LCD (Liquid Crystal Display), CRT (Cathode-Ray Tube) display, organic EL (Electronic Luminescence) display, inorganic EL display, plasma display, and the like. The display 17 displays the ultrasound image on the display screen according to the image signals output from the DSC 16.

For example, the controller 18 includes a CPU (Central Processing Unit), ROM (Read Only Memory), and RAM (Random Access Memory). Various process programs such as a system program stored in the ROM may be read out and deployed in the RAM, and the operation of each unit of the ultrasound diagnostic apparatus S may be centrally controlled according to the deployed program. The ROM includes a nonvolatile memory such as a semiconductor, and stores a system program corresponding to the ultrasound diagnostic apparatus S, various process programs which can be executed on the system program, and various types of data. Such programs may be stored in a format of a computer-readable program code and the CPU sequentially executes the process according to the program code. The RAM forms a work area temporarily storing the various programs executed by the CPU and the data used in such programs.

The ROM shall contain an ultrasound image display program to execute the ultrasound image display process described below. The controller 18 selects the first drive signal (third drive signal, fourth drive signal) and the second drive signal (fifth drive signal of symmetrical PI with the third drive signal, sixth drive signal of asymmetrical PI with the fourth drive signal) from several drive signals with different drive waveforms according to the ultrasound image display program. The controller 18 outputs the first and second drive signals by the transmitter 12 to transducers 2a of the ultrasound probe 2 in sequence on the same focusing line with a time delay so that the transmission ultrasound is focused at the same focal point. The controller 18 outputs the third and fifth drive signals to the first transducer group, which is in the inner side of the transmission aperture of transducer 2a, and outputs the fourth and sixth drive signals to the second transducer group, which is in the outer side of the transmission aperture, to cause the transmission ultrasound to be transmitted to the subject. Then, the controller 18 generates the reception signals of reception ultrasound corresponding to the third to sixth drive signals by the receiver 13, adds the reception signals corresponding to the first drive signal and the second drive signal by the image generator 14 by the pulse inversion method and extracts the higher harmonic wave component to generate the ultrasound image data, and causes the ultrasound image data to be displayed as an ultrasound image on the display 17 by the image processor 15 and DSC 16. The controller 18 performs a live image display that repeatedly generates and displays the ultrasound image data described above, and retains the ultrasound image data and stores it in the storage19 in response to the operation input of freeze and save from the user via the operation input unit 11 .

The storage 19 is a storage that can read and write information, such as HDD (Hard Disk Drive) and SSD (Solid State Drive), and stores information such as the ultrasound image data.

Next, FIG. 4A, FIG. 4B are used to explain the characteristics of the sound pressure of the ultrasound probe 2, which is a body cavity probe. FIG. 4A shows the transmission intensity of the transmission ultrasound for the channel of transducer 2a of the ultrasound probe 2. FIG. 4B shows the sound pressure rise contribution to the transmission center direction of the channel of transducer 2a of the ultrasound probe 2.

In the ultrasound probe 2, the acoustic radiation surface of transducer 2a is a small-diameter arc, which limits the increase in the transmission aperture width even if the number of elements of transducer 2a is increased. FIG. 4A shows that even if ultrasound is transmitted at the same transmission intensity for each channel of transducer 2a in the transmission aperture, as shown in FIG. 4B, there is less sound pressure rise contribution from ultrasound transmission in the channels outer side of the transmission aperture (away from the transmission center), due to the effect of the directional angle sensitivity. Therefore, backward shield (shadow) tolerance is weak if the transmission aperture’s transmission center is shielded by the acoustic shield.

Specific examples of acoustic shields that exhibit high reflection and high attenuation that cause shadows in the transvaginal echo area include uterine fibroids with calcification due to calcium accumulation and bubbles that have entered the cervical canal, while specific examples of high attenuation include uterine fibroids without calcification, malignant tumors, and cervical mucus.

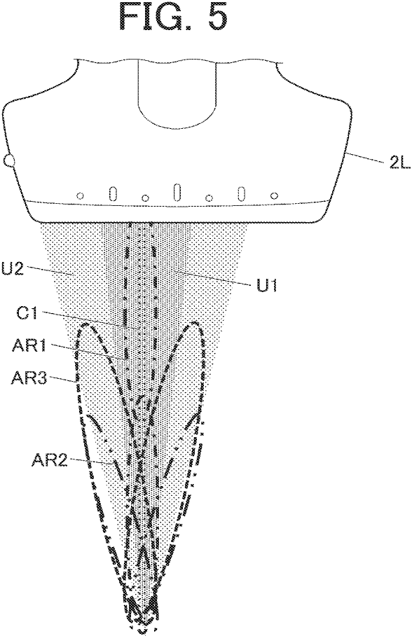

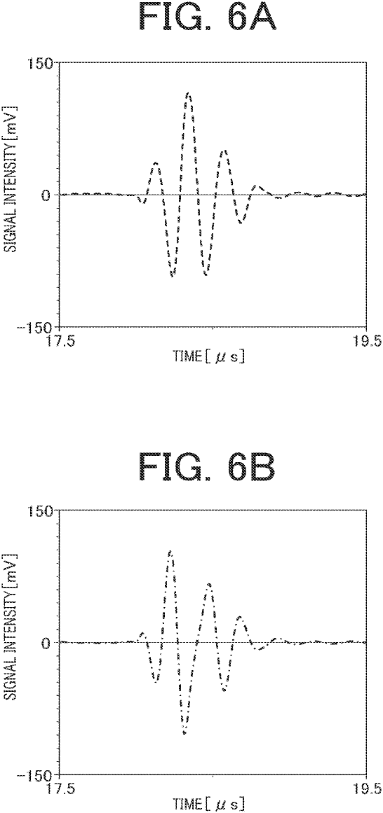

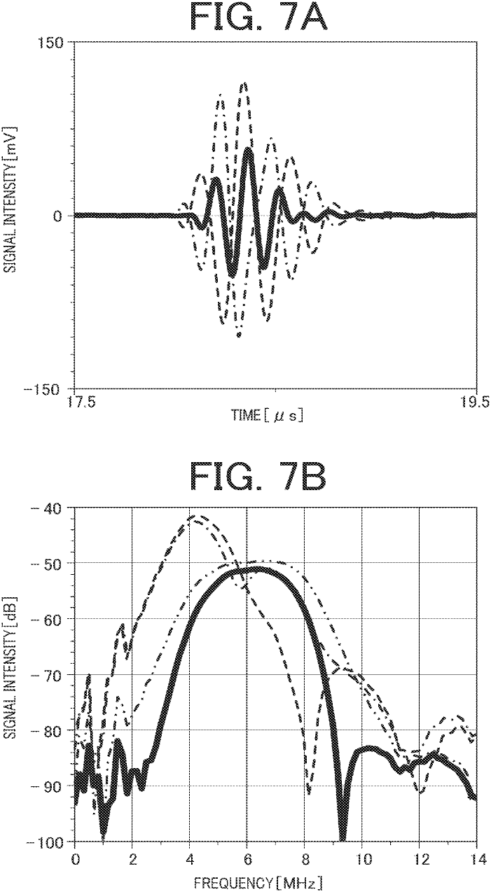

Next, FIG. 5 to FIG. 7B are used to refer to the pulse signals of the transmission ultrasound corresponding to the asymmetric PI drive signals of the present embodiment (the first drive signal (third drive signal + fourth drive signal) and the second drive signal (fifth drive signal + sixth drive signal) above). FIG. 5 shows the ultrasound beams corresponding to the first transducer group and the second dynamic group output from the ultrasound probe 2L. FIG. 6A shows an example of the time characteristic of the signal intensity of the transmission ultrasound corresponding to the fourth drive signal. FIG. 6B shows an example of the time characteristic of the signal intensity of the transmission ultrasound corresponding to the sixth drive signal. FIG. 7A shows an example of the time characteristics of the signal intensities of the transmission ultrasound corresponding to the fourth drive signal of FIG. 6A and the transmission ultrasound corresponding to the sixth drive signal of FIG. 6B, and their combined signal. FIG. 7B shows an example of the power spectra of the signal intensities of the transmission ultrasound corresponding to the fourth drive signal of FIG. 6A and the transmission ultrasound corresponding to the sixth drive signal of FIG. 6B, and their combined signal, and the transmission band of the ultrasound probe 2.

FIG. 5 uses the linear ultrasound probe 2L instead of the ultrasound probe 2 as the body cavity probe to illustrate the ultrasound beam clearly. The ultrasound beam generation of the ultrasound probe 2 is also the same as that of the ultrasound probe 2L.

As shown in FIG. 5, symmetrical PI drive signals (the third drive signal of the first drive signal and the fifth drive signal of the second drive signal) are input from the ultrasound probe 2L to the first transducer group, which is in the inner side of the transmission aperture, and symmetrical PI ultrasound beam U1 is output from the first transducer group, which is in the inner side, along the centerline C1 of the transmission center of the transmission aperture, so that it is focused to the focal point. Along with this, asymmetric PI drive signals (fourth drive signal of the first drive signal and sixth drive signal of the second drive signal) are input from the ultrasound probe 2L to the second transducer group, which is in the outer side of the transmission aperture, and asymmetric PI ultrasound beam U2 is output from the second transducer group in the outer side, outside the ultrasound beam U1, to be focused to the focal point as well. The sub-scanning of B-mode image generation causes the center line C1 to move in the scanning direction (the direction of the transducer array).

The area where the higher harmonic wave component of the ultrasound beam U1 is generated is shown by area AR1. The area where the higher harmonic wave component of the ultrasound beam U2 is generated is shown by area AR2. The area of offset residual generation of the combined signal of the asymmetric PI’s second and fourth pulse signals added (combined) is shown in area AR3.

Here is an example of the transmission ultrasound and combined signal of the asymmetric PI drive signal. As the pulse signal of the transmission ultrasound corresponding to the fourth drive signal, there is output the transmission ultrasound of the drive waveform (dashed line on the figure) of the signal intensity [mV] of the transmission ultrasound to the time [µs] shown in FIG. 6A. As the pulse signal of the transmission ultrasound corresponding to the sixth drive signal, there is output the transmission ultrasound of the drive waveform (single dotted line on the figure) of the signal intensity [mV] of the transmission ultrasound to the time [µs]shown in FIG. 6B. If these are reflected and obtained as the reception signals, the imaging signal obtained by calculating them is the signal obtained by adding (combining) the transmission ultrasound corresponding to the fourth drive signal in FIG. 6A and the transmission ultrasound corresponding to the sixth drive signal in FIG. 6B. Then, as shown in FIG. 7A, the signal intensity [mV] (the signal intensity of the combined signal is the solid line on the diagram) of the transmission ultrasound to the time [µs]of the combined signal of the transmission ultrasound corresponding to the fourth drive signal in FIG. 6A (dashed line) and the transmission ultrasound corresponding to the sixth drive signal in FIG. 6B (single dotted line) is obtained as the result of the calculation. In addition, by Fourier transforming the pulse signals of the transmission ultrasound corresponding to the fourth drive signal in FIG. 7A, transmission ultrasound corresponding to the sixth drive signal, and the transmission ultrasound of the combined signal, respectively, as shown in FIG. 7B, there is obtained the signal intensity[dB] (frequency power spectrum) of the transmission ultrasound with respect to the frequency [MHz] of the transmission ultrasound corresponding to the fourth drive signal, the transmission ultrasound corresponding to the sixth drive signal, and their combined signal. The component of the combined signal in FIGS. 7A and 7B is the offset residual.

As the first feature of the combined signal of asymmetric PI, from FIG. 7A, it is possible to obtain the offset residual of the asymmetric PI even at low sound pressure because it is a linear component, unlike the higher harmonic waves that occur in the high sound pressure area. As the second feature, as shown in FIG. 7B, from the reception signal corresponding to the low-frequency transmission ultrasound corresponding to the fourth drive signal of FIG. 6A and the low-frequency transmission ultrasound corresponding to the sixth drive signal of FIG. 6B, a broadband signal component can be obtained by adding together, making it possible to obtain the ultrasound image data with excellent distance resolution and speckle graininess. As the third feature, as shown in FIG. 7A transmission ultrasound of the fourth and sixth drive signals are low-frequency and therefore easily spread out (diffract easily) and go around the shadow portion of the acoustic shield, making them suitable for complementing the shadow portion.

In addition, FIG. 7B shows an example of the transmission band (transmission frequency band) of the ultrasound probe 2 (the signal intensity [dB] of the transmission ultrasound for frequency [MHz]) (two dotted lines on the figure). In the present embodiment and the comparative examples and examples described below, the ultrasound probe 2 of the transmission band characteristics of FIG. 7B shall be used. The transmission band of FIG. 7B is the transmission characteristic when a broadband impulse is input and shows the transmission band characteristics of the ultrasound probe 2, but the absolute values of the vertical axis (signal intensity [dB]) are for reference because the input energy is not equivalent.

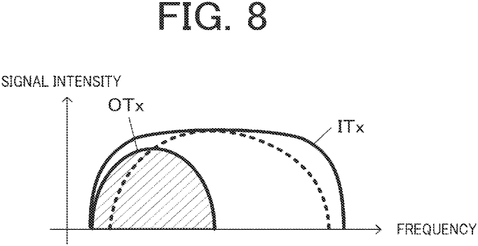

Next, FIG. 8 is used to explain the preferred conditions for the symmetric PI transmission ultrasound and the asymmetric PI transmission ultrasound. FIG. 8 shows the preferred power spectrum of the signal intensity of the transmission ultrasound of the first and second transducer groups.

FIG. 8 shows the -20 dB transmission frequency band of the ultrasound probe 2 (dashed line on the figure), the signal component ITx as the power spectrum (frequency power spectrum) of the signal intensity of the transmission ultrasound of the first transducer group, and the signal component OTx as the power spectrum (frequency power spectrum) of the signal intensity of the transmission ultrasound of the second transducer group.

First, condition (a) is that the central frequency of the signal component OTx < the central frequency of the signal component ITx. The central frequency ([MHz]) is the average of the upper and lower frequency limits that are first below -6 dB from the signal intensity value of the peak frequency of the frequency power spectrum.

Also, condition (b) is that the bandwidth of the signal component OTx < the bandwidth of the signal component ITx. The (frequency) bandwidth ([MHz]) is the difference between the upper and lower frequency limits that are first below -6 dB from the peak frequency intensity value of the frequency power spectrum. The fractional bandwidth [%] is the percentage of the above bandwidth divided by the above central frequency.

As condition (c), the signal intensity of the signal component OTx < the signal intensity of the signal component ITx in the higher frequency area than the -20 dB central frequency of the ultrasound probe 2.

It is known that the scatter intensity is proportional to the fourth power of the frequency when ultrasound waves are subjected to a scatterer that is sufficiently smaller than wavelength of the ultrasound waves. The configuration with the conditions (a), (b) and (c) is especially useful for improving the delineation of shallow no-to-low echo areas since it suppresses the higher harmonic wave component of the transmission waves from scattering into the living body tissue and entering the reception area as the acoustic noise. Furthermore, only the low frequency components necessary to obtain the sound pressure rise for higher harmonic wave generation in the vicinity of the transmission focal point will be delivered to the second transducer group, and the high frequency components that do not contribute to this will not be delivered. This reduces the amount of heat generated on the probe surface, and the like and improves transmission efficiency, which also improves penetration.

Also, as condition (d), the central frequency of the transmission ultrasound corresponding to the fourth drive signal of the first drive signal < the central frequency of the combined signal of the offset residual (offset residual component), and the central frequency of the transmission ultrasound corresponding to the sixth drive signal of the second drive signal < the central frequency of the offset residual component. For example, in FIG. 7B, condition (d) is satisfied.

Also, condition (e) is that the bandwidth of the transmission ultrasound corresponding to the fourth drive signal < the bandwidth of the offset residual component, and the bandwidth of the transmission ultrasound corresponding to the sixth drive signal < the bandwidth of the offset residual component. For example, in FIG. 7B, condition (e) is satisfied.

By designing the offset residual component so that conditions (d) and (e) are met, the amount of the transmission wave component that goes around the shadow portion of the acoustic shield in the ultrasound image is increased by taking advantage of the characteristics of the frequency component that is low (the shadow portion coverage effect is increased). The resulting offset residual component image signal has a higher frequency than that of the transmission ultrasound, and the wider band results in image delineation with finer image granularity and higher distance resolution.

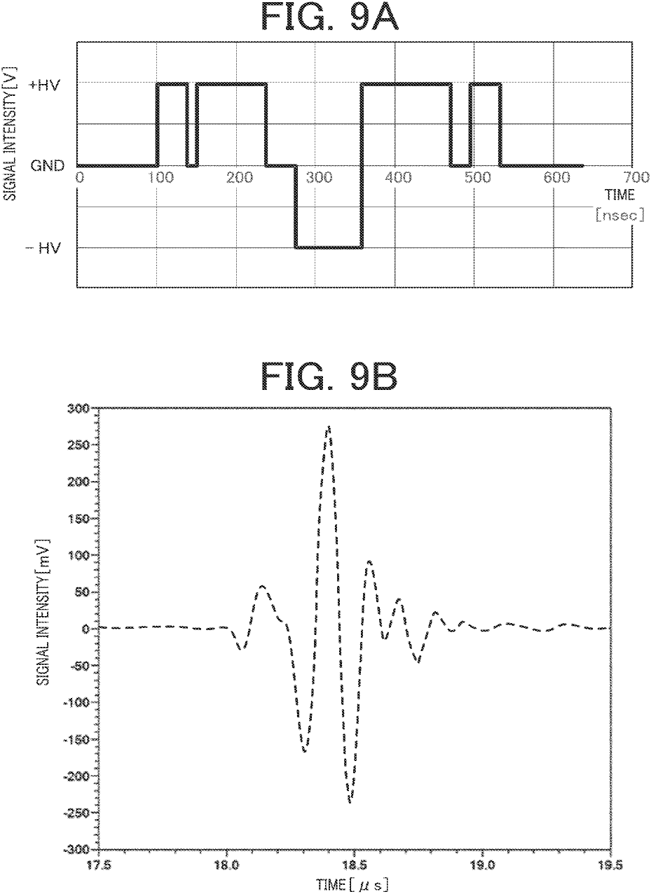

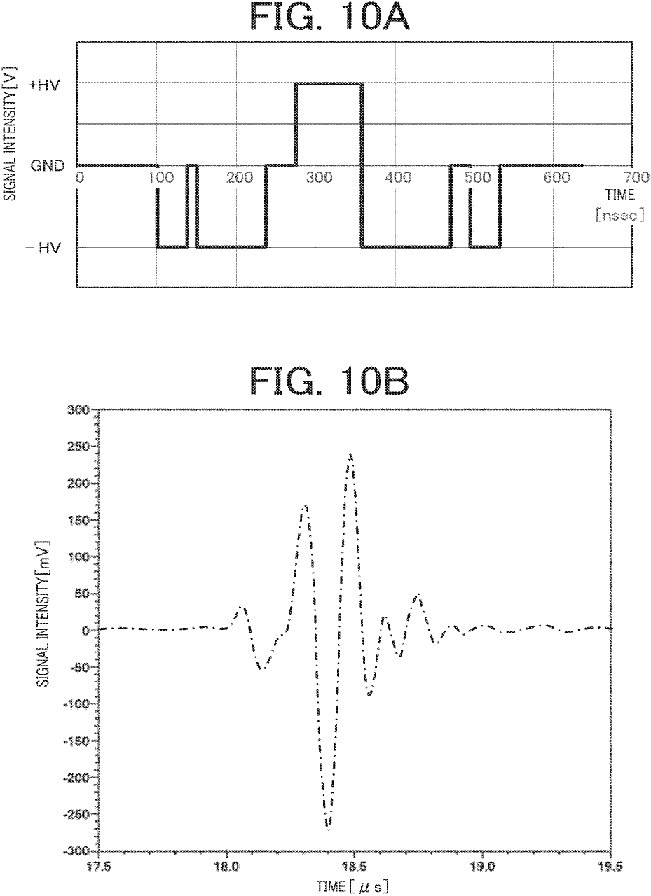

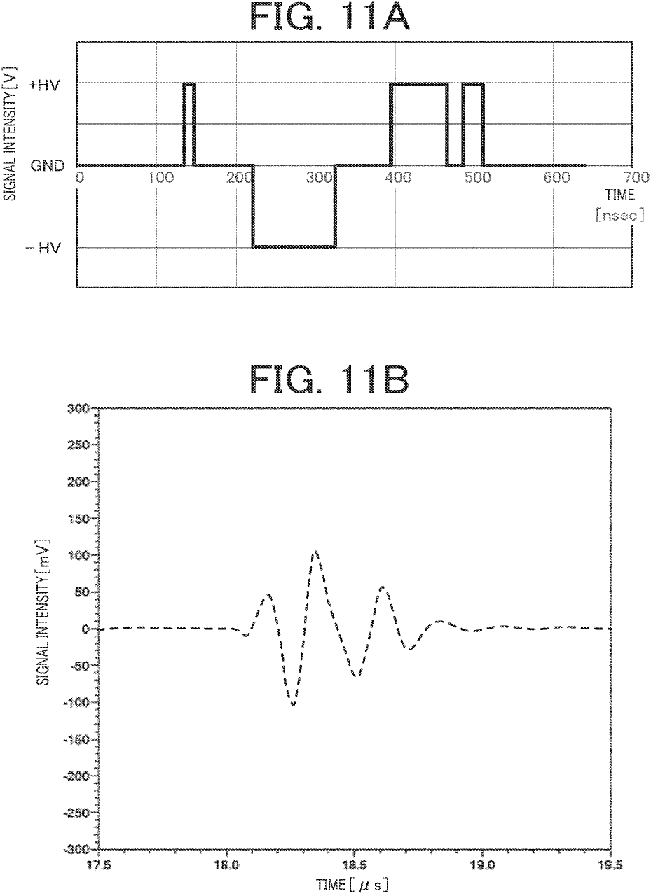

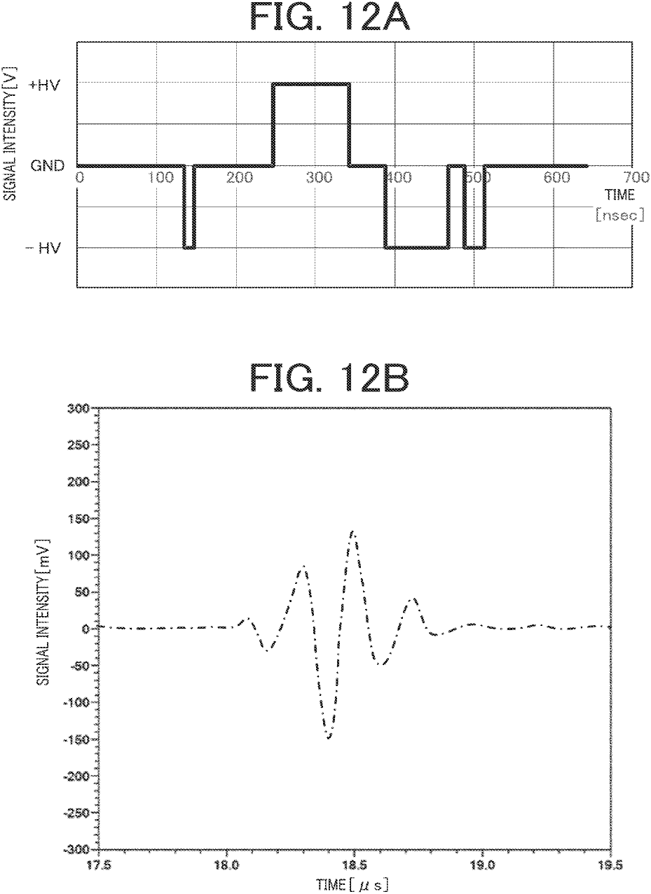

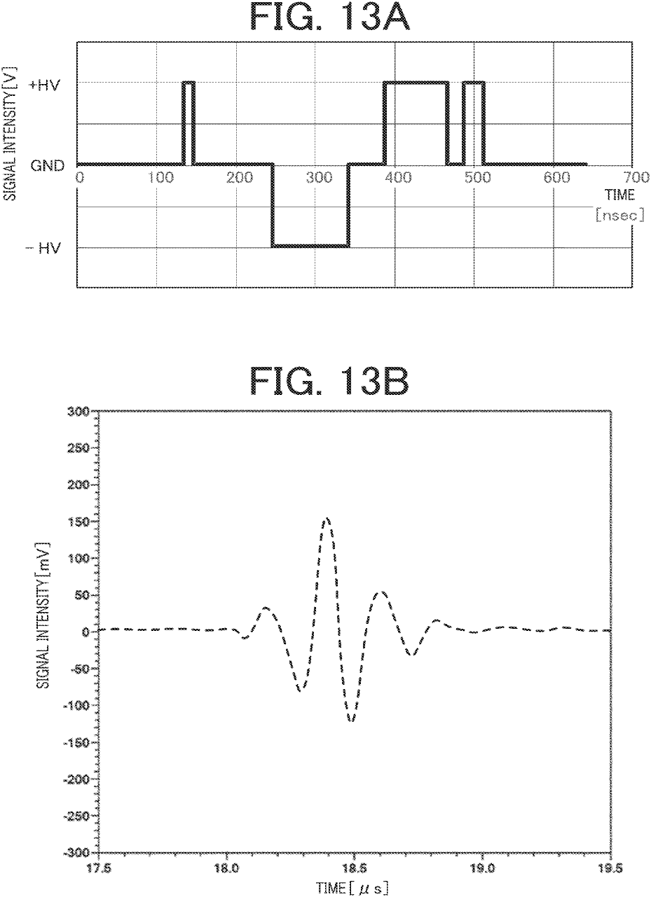

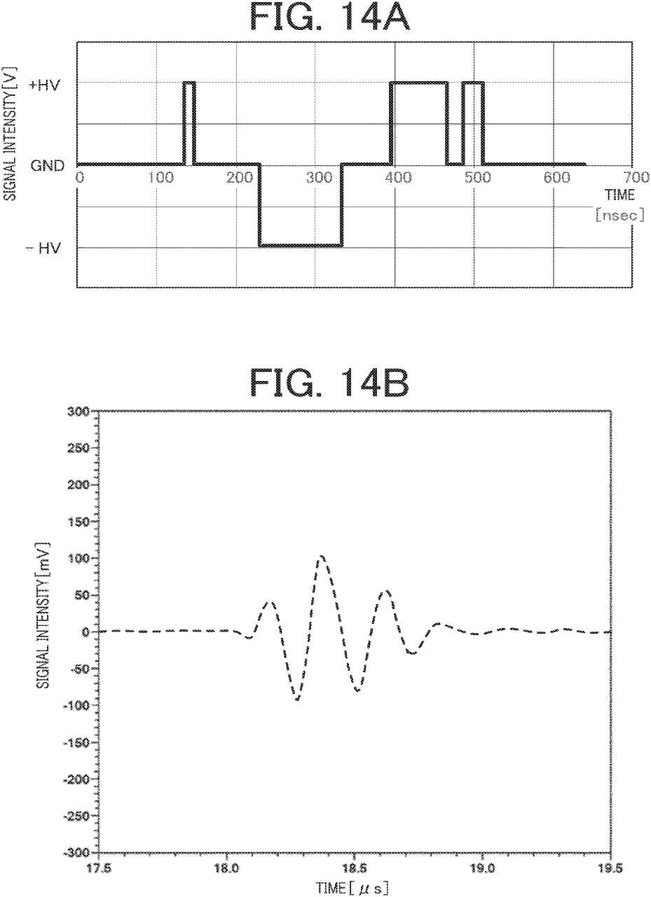

Next, FIG. 9A to FIG. 20 are used to explain the specific Comparative Examples 1-4 and Examples 1-3 with the ultrasound probe 2 and transmission conditions such as drive signals and the transmission ultrasound in the present embodiment. FIG. 9A shows the time characteristics of the signal intensity of the drive signal in drive waveform 1. FIG. 9B shows the time characteristics of the signal intensity of the transmission ultrasound corresponding to the drive signal of drive waveform 1. FIG. 10A shows the time characteristics of the signal intensity of the drive signal in drive waveform 2. FIG. 10B shows the time characteristics of the signal intensity of the transmission ultrasound corresponding to the drive signal in drive waveform 2. FIG. 11A shows the time characteristics of the signal intensity of the drive signal in drive waveform 3. FIG. 11B shows the time characteristics of the signal intensity of the transmission ultrasound corresponding to the drive signal in drive waveform 3. FIG. 12A shows the time characteristics of the signal intensity of the drive signal in drive waveform 4. FIG. 12B shows the time characteristics of the signal intensity of the transmission ultrasound corresponding to the drive signal in drive waveform 4. FIG. 13A shows the time characteristics of the signal intensity of the drive signal in drive waveform 5. FIG. 13B shows the time characteristics of the signal intensity of the transmission ultrasound corresponding to the drive signal in drive waveform 5. FIG. 14A shows the time characteristics of the signal intensity of the drive signal in drive waveform 6. FIG. 14B shows the time characteristics of the signal intensity of the transmission ultrasound corresponding to the drive signal in drive waveform 6.

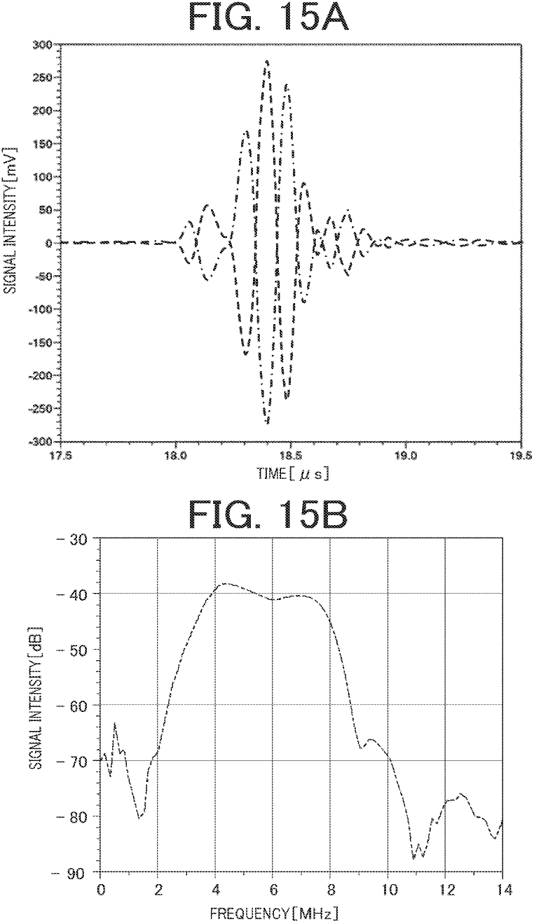

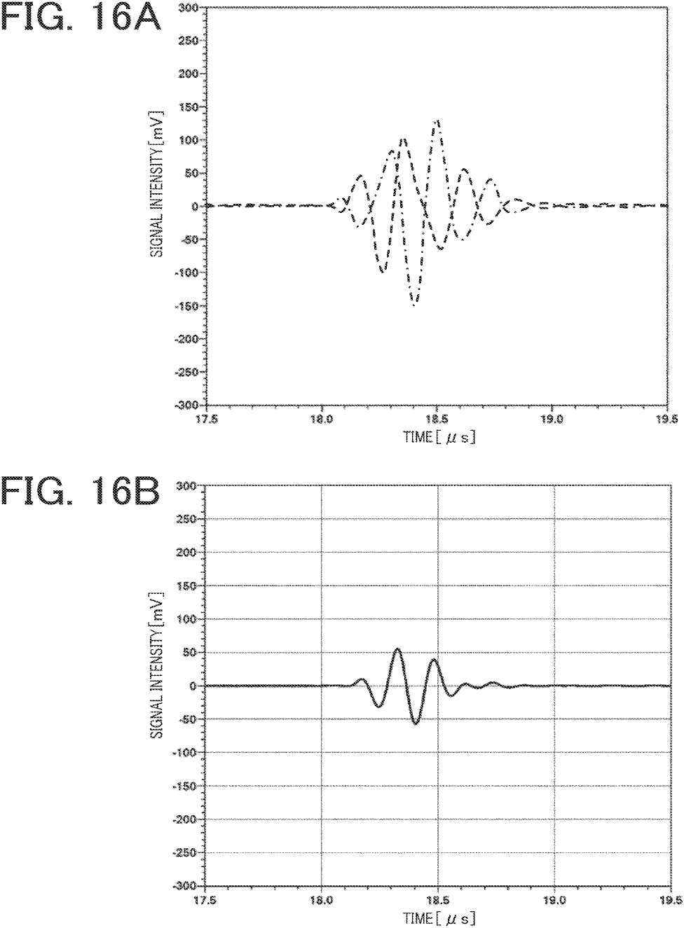

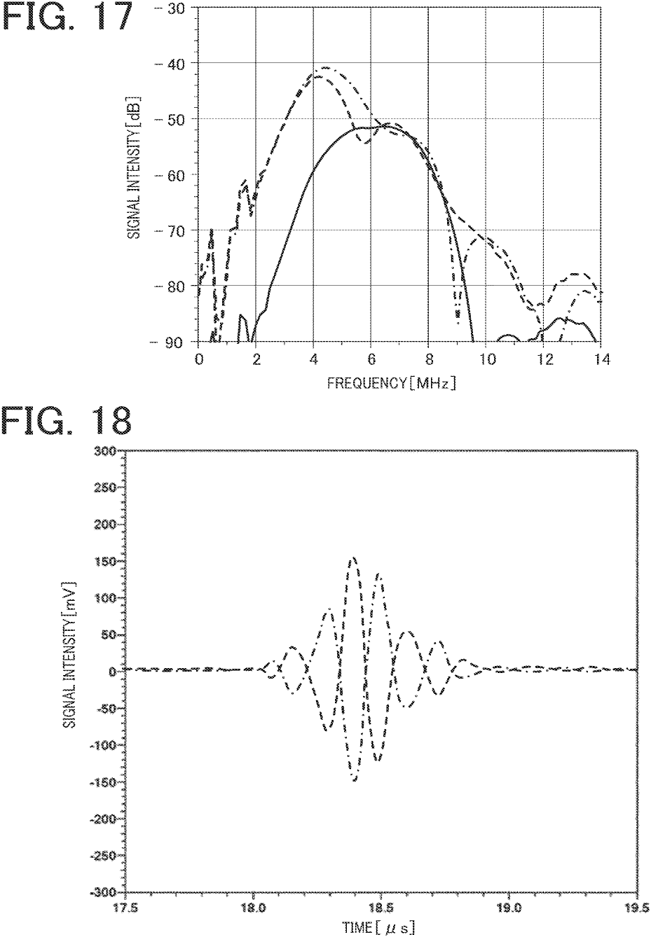

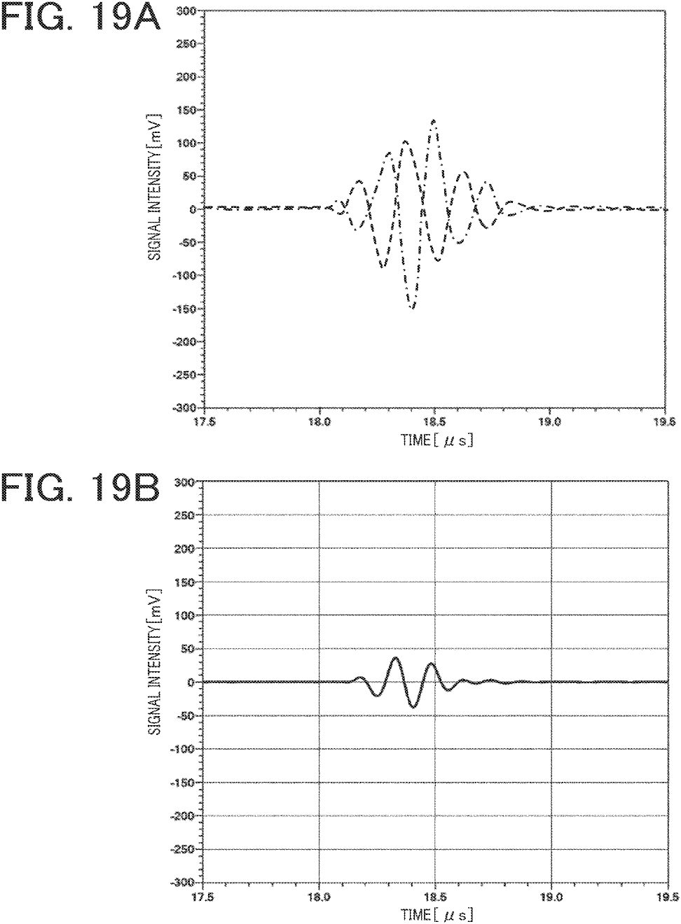

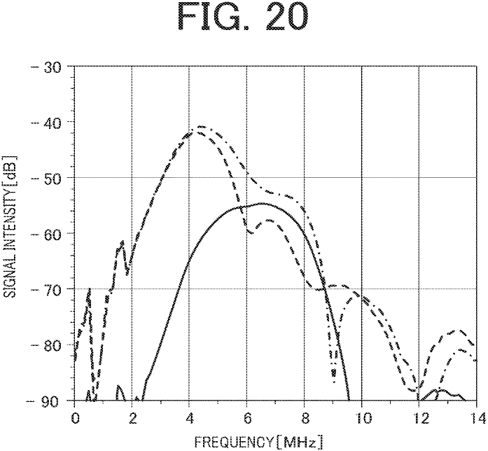

FIG. 15A shows the time characteristics of the signal intensity of the transmission ultrasound corresponding to the drive signals of drive waveforms 1 and 2. FIG. 15B shows the power spectra of the signal intensities of the transmission ultrasound corresponding to the drive signals of drive waveforms 1 and 2. FIG. 16A shows the time characteristics of the signal intensity of the transmission ultrasound corresponding to the drive signals of drive waveforms 3 and 4. FIG. 16B shows the time characteristics of the combined waveform 1 of the transmission ultrasound corresponding to the drive signals of drive waveforms 3 and 4. FIG. 17 shows the power spectra of the signal intensities of the transmission ultrasound corresponding to the drive signals of drive waveforms 3 and 4 and the combined signal of combined waveform 1. FIG. 18 shows the power spectra of the signal intensities of the transmission ultrasound corresponding to the drive signals of drive waveforms 5 and 4. FIG. 19A shows the time characteristics of the signal intensity of the transmission ultrasound corresponding to the drive signals of drive waveforms 6 and 4. FIG. 19B shows the time characteristics of the combined waveform 2 of the transmission ultrasound corresponding to the drive signals of drive waveforms 6 and 4. FIG. 20 shows the power spectra of the signal intensities of the transmission ultrasound corresponding to the drive signals of drive waveforms 6 and 4 and the combined signal of combined waveform 2.

The following is a description of the present invention with comparative examples and examples. The vertical axis of the transmission ultrasound time characteristics in the comparative examples and the examples is originally sound pressure (Pa), but for convenience, it is expressed as the voltage value (mV) measured with a hydrophone under the same conditions and with the same measurement system.

### <Comparative Example 1>

FIG. 9A to FIG. 10B, FIG. 15A and FIG. 15B are used for a description of Comparative Example 1. Comparative Example 1 does not divide the transducers 2a of the transmission aperture of the ultrasound probe 2 into a first transducer group, which is in the inner side at the center of the transmission aperture, and a second transducer group, which is in the outer side at the edge portion of the transmission aperture. The first drive signal of drive waveform 1 and the second drive signal of drive waveform 2 are output from the transmitter 12 to each transducer 2a of the transmission aperture in sequence, and the imaging signal obtained by the pulse inversion method based on the obtained reception signal is imaged in this comparative example.

A transvaginal probe with radius of curvature = 10 [mm] and element pitch of transducer 2a = 0.15 [mm] was used as the ultrasound probe 2. The transmission frequency bandwidth of the ultrasound probe 2 used is shown by the double-dashed line in FIG. 7B. In addition, as described below, image evaluation was performed with the transmission focal point of 30 [mm]. The transmission focal point of this the ultrasound probe 2 and image evaluation was common for Comparative Examples 1-4 and Examples 1-3. The number of elements of transducer 2a in the transmission aperture of the ultrasound probe 2 of Comparative Example 1 is 46.

The waveform of the first drive signal of Comparative Example 1 is designated as drive waveform 1. FIG. 9A shows the signal intensity [V] of the drive signal of drive waveform 1 with respect to time [ns]. FIG. 9B shows the signal intensity [mV] of the transmission ultrasound (the transmission ultrasound emitted by inputting the drive signal to the ultrasound probe 2) corresponding to the drive signal of drive waveform 1 with respect to time [µs]. In FIG. 9B, the signal intensity of the transmission ultrasound corresponding to drive waveform 1 is shown by the dashed line.

The second drive signal waveform of Comparative Example 1 is drive waveform 2. FIG. 10A shows the signal intensity [V] of the drive signal of drive waveform 2 with respect to time [ns]. FIG. 10B shows the signal intensity [mV] of the transmission ultrasound corresponding to the drive signal of drive waveform 2 with respect to time [µs]. In FIG. 10B, the signal intensity of the transmission ultrasound corresponding to drive waveform 2 is shown as a single dotted line.

FIG. 15A shows the superposition of signal intensity [mV] with respect to time [µs] between the transmission ultrasound corresponding to the drive signal in drive waveform 1 and the transmission ultrasound corresponding to the drive signal in drive waveform 2. FIG. 15B shows the superposition of signal intensity [dB] (frequency power spectrum) between the transmission ultrasound corresponding to the drive signal in drive waveform 1 and the transmission ultrasound corresponding to the drive signal in drive waveform 2, with respect to frequency [MHz]. In FIG. 15A and FIG. 15B, the signal intensity of the transmission ultrasound corresponding to drive waveform 1 is shown as a dashed line, and the signal intensity of the transmission ultrasound corresponding to drive waveform 2 is shown as a single dotted line. In 15B, the power spectra of drive waveform 1 and drive waveform 2 are shown superimposed because they are identical.

As shown in FIG. 15A signal intensity of the transmission ultrasound corresponding to drive waveform 2 is inverted in voltage polarity from the signal intensity of the transmission ultrasound corresponding to drive waveform 1. Therefore, as shown in FIG. 15B, the frequency power spectra of the transmission ultrasound corresponding to drive waveforms 1 and 2 coincide. Therefore, by adding the signal intensity of the transmission ultrasound corresponding to drive waveform 1 and the signal intensity of the transmission ultrasound corresponding to drive waveform 2, it is completely offset and no offset residual occurs.

As shown in FIG. 9A and FIG. 10A, drive signals for drive waveforms 1 and 2 are generated with three common values of GND (0 [V]), +HV, and -HV, simplifying the configuration and control of the transmitter 12. This applies to not only drive waveforms 1 and 2, but also drive waveforms 3 through 6.

The imaging signal of Comparative Example 1 is the addition signal (first-and-second transmission reception result addition signal) of a reception signal (first transmission reception result signal) generated at the receiver 13 by ultrasound transmission and reception corresponding to the first drive signal of drive waveform 1 output from the transmitter 12, and a reception signal (second transmission reception result signal) generated at the receiver 13 by ultrasound transmission and reception corresponding to the second drive signal of drive waveform 2 output from the transmitter 12. The higher harmonic wave component of the first-and-second transmission reception result addition signal is extracted by the higher harmonic wave component extractor 14a, and the image generator 14 uses said higher harmonic wave component to generate the ultrasound image data.

Although Cyst is well delineated because only higher harmonic wave components are used, shadows are strongly delineated because not enough higher harmonic waves are generated in areas where sound pressure is reduced due to shields.

### <Comparative Example 2>

FIG. 12A to FIG. 13B, and FIG. 18 are used to illustrate Comparative Example 2. In Comparative Example 2, the transducers 2a of the transmission aperture of the ultrasound probe 2 are divided into the first transducer group which is in the inner side of the transmission aperture and the second transducer group which is in the outer side of the transmission aperture, and, as the first drive signal, the third drive signal of drive waveform 1 is output from the transmitter 12 to the first transducer group in the inner side of the transmission aperture, and the fourth drive signal of drive waveform 2 is output from the transmitter 12 to the second transducer group in the outer side of the transmission aperture. Then, as the second drive signal, the fifth drive signal of drive waveform 5 is then output from the transmitter 12 to the first transducer group which is in the inner side of the transmission aperture, and the sixth drive signal of drive waveform 4 is output from the transmitter 12 to the second transducer group which is in the outer side of the transmission aperture. The imaging signal obtained by the pulse inversion method based on the reception signal obtained is imaged in the Comparative Example 2.

The number of elements of transducers 2a in the first transducer group which is in the inner side of the transmission aperture of the ultrasound probe 2 of the Comparative Example 2 is 16, and the number of elements of transducers 2a in the second transducer group which is in the outer side is 30.

The waveform of the third drive signal output to the first transducer group which is in the inner side of the transmission aperture, of the first drive signal of Comparative Example 2, is drive waveform 1. The waveform of the fifth drive signal that is input to the first transducer group, which is in the inner side of the transmission aperture, of the second drive signal of Comparative Example 2 is drive waveform 2.

The waveform of the fourth drive signal output to the second transducer group, which is in the outer side of the transmission aperture, of the first drive signal of Comparative Example 2 is the drive waveform 5. FIG. 13A shows the signal intensity [V] of the drive signal of drive waveform 5 with respect to time [ns]. FIG. 13B shows the signal intensity [mV] of the transmission ultrasound corresponding to the drive signal of drive waveform 5 with respect to time [µs]. In FIG. 13B, the signal intensity of the transmission ultrasound corresponding to drive waveform 5 is shown by the dashed line.

The waveform of the sixth drive signal output to the second transducer group which is in the outer side of the transmission aperture, of the second drive signal of Comparative Example 2 is the drive waveform 4. FIG. 12A shows the signal intensity [V] of the drive signal of drive waveform 4 with respect to time [ns]. FIG. 12B shows the signal intensity [mV] of the transmission ultrasound corresponding to the drive signal of drive waveform 4 with respect to time [µs]. In FIG. 12B, the signal intensity of the transmission ultrasound corresponding to drive waveform 4 is shown as a single dotted line.

For the addition of the transmission ultrasound between the third drive signal of drive waveform 1 and the fifth drive signal of drive waveform 2 in Comparative Example 2, there is no offset residual as in Comparative Example 1. Also, no offset residuals occur for the addition of the transmission ultrasound with the third drive signal of drive waveform 5 and the fifth drive signal of drive waveform 4 in Comparative Example 2.

FIG. 18 shows the superposition of signal intensity [mV] with respect to time [µs] between the transmission ultrasound corresponding to the drive signal in drive waveform 5 and the transmission ultrasound corresponding to the drive signal in drive waveform 4. In FIG. 18, the signal intensity of the transmission ultrasound corresponding to drive waveform 5 is shown as a dashed line, and the signal intensity of the transmission ultrasound corresponding to drive waveform 4 is shown as a single dotted line.

As shown in FIG. 18, the signal intensity of the transmission ultrasound corresponding to drive waveform 4 is inverted in voltage polarity with the signal intensity of the transmission ultrasound corresponding to drive waveform 5. Therefore, the frequency power spectra of the transmission ultrasound corresponding to drive waveforms 5 and 4 are matched. Therefore, by adding the signal intensity of the transmission ultrasound corresponding to drive waveform 5 and the signal intensity of the transmission ultrasound corresponding to drive waveform 4, it is completely offset and no offset residual occurs.

The imaging signal of Comparative Example 2 is an addition signal (first-and-second transmission reception result addition signal) of the reception signal (first transmission reception result signal) and the reception signal (second transmission reception result signal). The reception signal (first transmission reception result signal) is generated at the receiver 13 by transmission and reception of ultrasound corresponding to the third drive signal of drive waveform 1 output from the first transducer group which is in the inner side of the transmission aperture of the transmitter 12 and the ultrasound corresponding to the fourth drive signal of drive waveform 5 output from the second transducer group which is in the outer side of the transmission aperture. The reception signal (second transmission reception result signal) is generated at the receiver 13 by transmission and reception of ultrasound corresponding to the fifth drive signal of drive waveform 2 output from the first transducer group which is in the inner side of the transmission aperture of the transmitter 12, and the ultrasound corresponding to the sixth drive signal of drive waveform 4 output from the second transducer group which is in the outer side of the transmission aperture. The higher harmonic wave component of the first-and-second transmission reception result addition signal is extracted by the higher harmonic wave component extractor 14a, and the image generator 14 uses said higher harmonic wave component to generate the ultrasound image data.

The frequency power spectrum of the ultrasound wave output from the first transducer group is shown in FIG. 15B, and the frequency power spectrum of the ultrasound wave output from the second transducer group is shown by the single dotted line in FIG. 17. The drive waveforms 4 and 5 are polarity-inversed symmetrical and the same frequency power spectrum. Thus, these are in the preferred relationship shown in FIG. 8. Therefore, the transmission efficiency is improved compared to Comparative Example 1, and in addition to good Cyst delineation, penetration is also improved. However, since only the higher harmonic wave component is still used, not enough higher harmonic waves are generated in the areas where sound pressure is reduced due to shielding, and shadows are strongly delineated.

### < Comparative Example 3>

Comparative Example 3 is explained. In Comparative Example 3, the same transmission conditions as Comparative Example 2 are used to output the first and second drive signals (third to sixth drive signals) from the transmitter 12 to the transducers 2a of the transmission aperture in the ultrasound probe 2. The imaging signals obtained by the pulse inversion method based on the reception signals obtained and the imaging signals obtained by the reception signals of second transmission are combined to create an image in Comparative Example 3.

The imaging signal of Comparative Example 3 is a composite image obtained from 50% of the image obtained from, as the imaging signal, the first-and-second transmission reception result addition signal which is the addition signal of the first transmission reception result signal and the second transmission reception result signal in Comparative Example 2 and 50% of the image obtained from the second transmission reception result signal as the imaging signal. In other words, the higher harmonic wave component is extracted by the higher harmonic wave component extractor 14a using the first-and-second transmission reception result addition signal, and, by the image generator 14, the ultrasound image data generated using the higher harmonic wave component is combined with the image data generated without the higher harmonic wave component extraction process from the second transmission reception result signal, each with a weighting of 50%.

In addition to the higher harmonic wave component, the fundamental wave component used as the second transmission is also used for imaging. Therefore, even in shadow parts where sound pressure is reduced, the fundamental wave component provides the imaging component and reduces shadows, but the contrast improvement effect of the higher harmonic wave is not obtained, and the effect of acoustic noise becomes larger in low brightness areas, resulting in a gradual tonal change in the boundary of the shallow Cyst (shallow portion Cyst) delineation, degrading visibility and reducing its area size.

### <Comparative Example 4>

FIG. 11A, FIG. 11B, and FIG. 16A through FIG. 17 are used to explain Comparative Example 4. Comparative Example 4 does not divide the transducers 2a of the transmission aperture of the ultrasound probe 2 into the first transducer group in the inner side and the second transducer group in the outer side. The first drive signal of drive waveform 3 and the second drive signal of drive waveform 4 are output from the transmitter 12 to each transducer 2a of the transmission aperture in sequence, and imaging signals obtained by the reception signal addition operation according to the pulse inversion method based on the obtained the reception signals are imaged in Comparative Example 4.

The waveform of the first drive signal of the Comparative Example 4 is the drive waveform 3. FIG. 11A shows the signal intensity [V] of the drive signal of drive waveform 3 with respect to time [ns]. FIG. 11B shows the signal intensity [mV] of the transmission ultrasound corresponding to the drive signal of drive waveform 3 with respect to time [µs]. In FIG. 11B, the signal intensity of the transmission ultrasound corresponding to drive waveform 3 is shown by the dashed line.

The second drive signal waveform of Comparative Example 4 is drive waveform 4.

FIG. 16A shows the superposition of signal intensity [mV] with respect to time [µs] between the transmission ultrasound corresponding to the drive signal in drive waveform 3 and the transmission ultrasound corresponding to the drive signal in drive waveform 4. FIG. 16B shows the signal intensity [mV] of the combined signal of the combined waveform 1 of the transmission ultrasound corresponding to the drive signal of drive waveform 3 and the transmission ultrasound corresponding to the drive signal of drive waveform 4, with respect to time [µs]. FIG. 17 shows superposition of the signal intensity [dB] (frequency power spectrum) of the combined signal in combined waveform 1 of the transmission ultrasound corresponding to the drive signal in drive waveform 3 and the transmission ultrasound corresponding to the drive signal in drive waveform 4, with respect to frequency [MHz]. In FIG. 16A to FIG. 17, the signal intensity of the transmission ultrasound corresponding to drive waveform 3 is shown as a dashed line, the signal intensity of the transmission ultrasound corresponding to drive waveform 4 is shown as a single dotted line, and the signal intensity of the combined signal of combined waveform 1 is shown as a solid line.

As shown in 16A, the signal intensity of the transmission ultrasound corresponding to drive waveform 4 is not perfect, although the polarity of the voltage and the signal intensity of the transmission ultrasound corresponding to drive waveform 3 is roughly inverted. Therefore, as shown in FIG. 16B, the combined signal in combined waveform 1 of the transmission ultrasound corresponding to drive waveforms 3 and 4 does not become zero, but becomes an offset residual. In addition, as shown in FIG. 17, the frequency power spectra of the transmission ultrasound corresponding to drive waveforms 3 and 4 and the combined signal of combined waveform 1 do not match.

The imaging signal of Comparative Example 4 is the addition signal (first-and-second transmission reception result addition signal) of a reception signal (first transmission reception result signal) generated at the receiver 13 by ultrasound transmission and reception corresponding to the first drive signal of drive waveform 3 output from the transmitter 12, and a reception signal (second transmission reception result signal) generated at the receiver 13 by ultrasound transmission and reception corresponding to the second drive signal of drive waveform 4 output from the transmitter 12. The higher harmonic wave component extractor 14a extracts the higher harmonic wave component of the first-and-second transmission reception result addition signal as well as the offset residual component, and the image generator 14 generates the ultrasound image data using the higher harmonic wave component and the offset residual component.

In addition to the higher harmonic wave component, the offset residual component, which is a linear component, is used for imaging. Therefore, even in shadow parts where sound pressure has decreased, the fundamental wave component provides the imaging component and reduces shadows. However, the effect of contrast improvement by higher harmonic waves is not obtained, and the effect of acoustic noise increases in low brightness areas, resulting in a gradual tonal change in the boundary of the shallow Cyst delineation, degrading visibility and reducing its area size.

### <Example 1>

Example 1 is explained. In Example 1, the transducers 2a of the transmission aperture of the ultrasound probe 2 are divided into the first transducer group which is in the inner side and the second transducer group which is in the outer side, and, as the first drive signal, the third drive signal of drive waveform 1 is output from the transmitter 12 to the first transducer group in the inner side, and the fourth drive signal of drive waveform 3 is output from the transmitter 12 to the second transducer group in the outer side. Then, as the second drive signal, the fifth drive signal of drive waveform 2 is then output from the transmitter 12 to the first transducer group in the inner side, and the sixth drive signal of drive waveform 4 is output from the transmitter 12 to the second transducer group which is in the outer side. The imaging signal obtained by the reception signal addition operation according to the pulse inversion method based on the reception signal obtained is imaged in Example 1.

The number of elements of transducers 2a in the first transducer group, which is in the inner side of the transmission aperture of the ultrasound probe 2 of Example 1, is 16, and the number of elements of transducers 2a in the second transducer group, which is in the outer side, is 30.

The waveform of the third drive signal output to the first transducer group on the inner side, of the first drive signal of Example 1, is designated as drive waveform 1. The waveform of the fourth drive signal output to the first transducer group in the inner side of the second drive signal of Example 1 shall be drive waveform 2.

The waveform of the fourth drive signal output to the second transducer group which is in the outer side, of the first drive signal of Example 1, is designated as drive waveform 3. The waveform of the sixth drive signal output to the second transducer in the outer side, of the second drive signal of Example 1, is designated as drive waveform 4.

For the addition of the transmission ultrasound between the third drive signal of drive waveform 1 and the fifth drive signal of drive waveform 2 in Example 1, there is no offset residual as in Comparative Example 1. The offset residual of the combined signal of combined waveform 1 occurs for the addition of the transmission ultrasound between the forth drive signal of drive waveform 3 and the sixth drive signal of drive waveform 4 in Example 1.

The imaging signal of Example 1 is an addition signal (first-and-second transmission reception result addition signal) of the reception signal (first transmission reception result signal) and the reception signal (second transmission reception result signal). The reception signal (first transmission reception result signal) is generated at the receiver 13 by transmission and reception of ultrasound corresponding to the third drive signal of drive waveform 1 output from the first transducer group which is in the inner side of the transmitter 12 and the ultrasound corresponding to the fourth drive signal of drive waveform 3 output from the second transducer group which is in the outer side. The reception signal (second transmission reception result signal) is generated at the receiver 13 by transmission and reception of ultrasound corresponding to the fifth drive signal of drive waveform 2 output from the first transducer group which is in the inner side of the transmitter 12, and the ultrasound corresponding to the sixth drive signal of drive waveform 4 output from the second transducer group which is in the outer side. The higher harmonic wave component of the first-and-second transmission reception result addition signal is extracted and the offset residual component is also extracted by the higher harmonic wave component extractor 14a, and the image generator 14 uses said higher harmonic wave component and the offset residual component to generate the ultrasound image data.

The first transducer group, which does not produce the offset residual component, has a large proportion and is almost unaffected by shallow acoustic noise (shallow portion acoustic noise), resulting in good shallow Cyst delineation, while deep portion shadow areas are sufficiently improved since the image signal is obtained by the offset residual component obtained by the second transducer group. For shallow portion shadows, the image signal from the second transducer group is slightly weak, but improves to a level that is acceptable for practical use.

Furthermore, the frequency power spectrum of the ultrasound wave output from the first transducer group is as shown in FIG. 15B, and the frequency power spectrum of the ultrasound output from the second transducer group is shown by the dashed and single dotted lines in FIG. 17, and thus, these are the preferred relationships shown in FIG. 8. Therefore, penetration is also good.

### <Example 2>

Example 2 is described. In Example 2, the same drive signals (third to sixth drive signals) as in Example 1 are used, and the total number of elements in the transmission aperture is also the same, but the number of elements of transducer 2a in the first transducer group, which is in the inner side of the transmission aperture, is 10 and the number of elements of transducer 2a of the second transducer group, which is in the outer side, is 36, in Example 2

Compared to Example 1, the percentage of the first transducer group that does not produce offset residual components is reduced, and although there is some influence from shallow acoustic noise, the shallow Cyst delineation is at a level acceptable for practical use, and shallow portion shadows are improved since the image signal by the second transducer group is more obtained. The improvement effect on the deep portion shadow areas is generally the same as in Example 1, and the relationship between the frequency power spectrum of the ultrasound waves output from the first transducer group and the frequency power spectrum of the ultrasound waves output from the second transducer group is also the preferred relationship shown in FIG. 8 as in Example 1, and thus the penetration is also good.

### <Example 3>

FIG. 14A, FIG. 14B, FIG. 19A through FIG. 20 are used to illustrate Example 3. In Example 3, the transducers 2a of the transmission aperture of the ultrasound probe 2 are divided into the first transducer group, which is the inner side, and the second transducer group, which is in the outer side, and the third drive signal of the drive waveform 1 is output as the first drive signal to the first transducer group which is in the inner side from the transmitter 12. The fourth drive signal of drive waveform 6 is output from the transmitter 12 to the second transducer group which is in the outer side. Then, as the second drive signal, the fifth drive signal of drive waveform 2 is output from the transmitter 12 to the first transducer group which is in the inner side, and the sixth drive signal of drive waveform 4 is output from the transmitter 12 to the second transducer group which is in the outer side. The imaging signals obtained by the reception signal addition operation according to the pulse inversion method based on the obtained the reception signals are imaged in Example 3.

The number of elements of transducers 2a in the first transducer group, which is in the inner side of the transmission aperture of the ultrasound probe 2 of Example 3, is 10, and the number of elements of transducers 2a in the second transducer group, which is in the outer side, is 36.

The waveform of the third drive signal output to the first transducer group in the inner side, of the first drive signal of Example 3, is designated as drive waveform 1. The waveform of the fifth drive signal output to the first transducer group in the inner side of the second drive signal of Example 3 is drive waveform 2.

The waveform of the fourth drive signal output to the second transducer group in the outer side, of the first drive signal of Example 3, is designated as drive waveform 6. FIG. 14A shows the signal intensity [V] of the drive signal of drive waveform 6 with respect to time [ns]. FIG. 14B shows the signal intensity [mV] of the transmission ultrasound corresponding to the drive signal of drive waveform 6 with respect to time [µs]. In 14B, the signal intensity of the transmission ultrasound corresponding to drive waveform 6 is shown by the dashed line.

The waveform of the sixth drive signal output to the second transducer in the outer side, of the second drive signal of Example 3, is designated as drive waveform 4.

For the addition of the transmission ultrasound between the third drive signal of drive waveform 1 and the fifth drive signal of drive waveform 2 in Example 3, there is no offset residual as in Comparative Example 1. The offset residual occurs for the addition of the transmission ultrasound between the third drive signal of drive waveform 6 and the fifth drive signal of drive waveform 4 in Example 3.

FIG. 19A shows the superposition of signal intensity [mV] with respect to time [µs] between the transmission ultrasound corresponding to the drive signal in drive waveform 6 and the transmission ultrasound corresponding to the drive signal in drive waveform 4. FIG. 19B shows the signal intensity [mV] of the combined signal of the combined waveform 2 of the transmission ultrasound corresponding to the drive signal of drive waveform 6 and the transmission ultrasound corresponding to the drive signal of drive waveform 4, with respect to time [µs]. FIG. 20 shows superposition of the signal intensity [dB] (frequency power spectrum) of the combined signal in combined waveform 2 of the transmission ultrasound corresponding to the drive signal in drive waveform 6 and the transmission ultrasound corresponding to the drive signal in drive waveform 4, with respect to frequency [MHz]. In FIG. 19A to FIG. 20, the signal intensity of the transmission ultrasound corresponding to drive waveform 6 is shown as a dashed line, the signal intensity of the transmission ultrasound corresponding to drive waveform 4 is shown as a single dotted line, and the signal intensity of the combined signal of combined waveform 2 is shown as a solid line.

As shown in FIG. 19A signal intensity of the transmission ultrasound corresponding to drive waveform 4 is not perfect, although the polarity of the voltage and the signal intensity of the transmission ultrasound corresponding to drive waveform 6 is roughly inverted. Therefore, as shown in FIG. 19B, the combined signal in combined waveform 1 of the transmission ultrasound corresponding to drive waveforms 6 and 4 does not become zero, but becomes an offset residual. In addition, as shown in FIG. 20, the frequency power spectra of the transmission ultrasound corresponding to drive waveforms 6 and 4 and the combined signal of combined waveform 2 do not match. In addition, the frequency power spectrum of the combined waveform 2 shown by the solid line in FIG. 20 shows that the frequency bandwidth and central frequency of the combined waveform are similar to the frequency power spectrum of the combined waveform 1 shown by the solid line in FIG. 17, but its component intensities are low overall, and the offset residual component intensity of the combined waveform 2 is weaker than that of the combined waveform 1.

The imaging signal of Example 3 is an addition signal (first-and-second transmission reception result addition signal) of the reception signal (first transmission reception result signal) and the reception signal (second transmission reception result signal). The reception signal (first transmission reception result signal) is generated at the receiver 13 by transmission and reception of ultrasound corresponding to the third drive signal of drive waveform 1 output from the first transducer group which is in the inner side of the transmitter 12 and the ultrasound corresponding to the fourth drive signal of drive waveform 6 output from the second transducer group which is in the outer side. The reception signal (second transmission reception result signal) is generated at the receiver 13 by transmission and reception of ultrasound corresponding to the fifth drive signal of drive waveform 2 output from the first transducer group which is in the inner side of the transmitter 12, and the ultrasound corresponding to the sixth drive signal of drive waveform 4 output from the second transducer group which is in the outer side. The higher harmonic wave component of the first-and-second transmission reception result addition signal is extracted and the offset residual component is also extracted by the higher harmonic wave component extractor 14a, and the image generator 14 uses said higher harmonic wave component and the offset residual component to generate the ultrasound image data.

Compared to Example 2 having the same numbers of elements in the first and second transducer groups, the shallow acoustic noise effect is almost eliminated and the shallow Cyst is better delineated because the offsetting residual component obtained by the second transducer group is reduced. The shadow reduction effect is similar to that of Example 2, and the first transducer group, which does not cause offset residuals, has a smaller percentage, so the effect of going around to the shallow shadow parts is sufficient. However, the total offset residual component is reduced, so there is no practical problem in deep portion, but the level is slightly lower. The relationship between the frequency power spectrum of the ultrasound wave output from the first transducer group and the frequency power spectrum of the ultrasound wave output from the second transducer group is also in the preferred relationship shown in FIG. 8 as in Example 1, so the penetration is also good.

The transmission conditions (the transmission ultrasound) for Examples 1-3 are assumed to meet conditions (a) through (e).

### <Image Evaluation>

Based on the transmission conditions and imaging signals of Comparative Examples 1-4 and Examples 1-3, the ultrasound image data was generated by the ultrasound diagnostic apparatus S, and image evaluation of the generated the ultrasound image data was performed. The transmission conditions, imaging signals and image evaluations for Comparative Examples 1-4 and Examples 1-3 are shown in Table II below.

TABLE 2

TRANSMISSION CONDITION

| | FIRST DRIVE SIGNAL | | SECOND DRIVE SIGNAL | | TRANSMISSION APERTURE (NUMBER OF ELEMENTS) | | OFFSET RESIDUAL |
|---|---|---|---|---|---|---|---|
| | FIRST TRANSDUCER GROUP | SECOND TRANSDUCER GROUP | FIRST TRANSDUCER GROUP | SECOND TRANSDUCER GROUP | FIRST TRANSDUCER GROUP | SECOND TRANSDUCER GROUP | |
| COMPARATIVE EXAMPLE 1 | DRIVE WAVEFORM 1 | | DRIVE WAVEFORM 2 | | 46 | | ABSENT |
| COMPARATIVE EXAMPLE 2 | DRIVE WAVEFORM 1 | DRIVE WAVEFORM 5 | DRIVE WAVEFORM 2 | DRIVE WAVEFORM 4 | 16 | 30 | ABSENT |
| COMPARATIVE EXAMPLE 3 | DRIVE WAVEFORM 1 | DRIVE WAVEFORM 5 | DRIVE WAVEFORM 2 | DRIVE WAVEFORM 4 | 16 | 30 | ABSENT |
| COMPARATIVE EXAMPLE 4 | DRIVE WAVEFORM 3 | | DRIVE WAVEFORM 4 | | 46 | | PRESENT ((COMBINED WAVEFORM 1)) |
| EXAMPLE 1 | DRIVE WAVEFORM 1 | DRIVE WAVEFORM 3 | DRIVE WAVEFORM: 2 | DRIVE WAVEFORM 4 | 16 | 30 | PRESENT ((EDGE PORTION ONLY)) ((COMBINED WAVEFORM 1)) |
| EXAMPLE 2 | DRIVE WAVEFORM 1 | DRIVE WAVEFORM 3 | DRIVE WAVEFORM 2 | DRIVE WAVEFORM 4 | 10 | 36 | PRESENT ((EDGE PORTION ONLY)) ((COMINNED WAVEFORM 1)) |
| EXAMPLE 3 | DRIVE WAVEFORM 1 | DRIVE WAVEFORM 6 | DRIVE WAVEFORM 2 | DRIVE WAVEFORM 4 | 10 | 36 | PRESENT ((EDGE PORTION ONLY)) ((COMBINED WAVEFORM 2)) |

| | IMAGING SIGNAL | IMAGE EVALUATION | | | |
|---|---|---|---|---|---|
| | | SHALLOW CYST DELINEATION | SHADOW REDUCTION EFFECT | | PENETRATION |
| | | | SHALLOW PORTION | DEEP PORTION | |
| COMPARATIVE EXAMPLE 1 | FIRST -AND-SECOND TRANSMISSION RECEPTION RESULT ADDITION SIGNAL | O | × | × | Δ |
| COMPARATIVE EXAMPLE 2 | FIRST-AND-SECOND TRANSMISSION RECEPTION RESULT ADDITION SIGNAL | O | × | × | O |
| COMPARATIVE EXAMPLE 3 | FIRST-AND-SECOND TRANSMISSION RECEPTION RESULT ADDITION SIGNAL (50%) + SECOND TRANSMISSION RECEPTION SIGNAL (50%) | × | O | O | O |
| COMPARATIVE EXAMPLE 4 | FIRST-AND-SECOND TRANSMISSION RECEPTION RESULT ADDITION SIGNAL | × | O | O | O |
| EXAMPLE 1 | FIRST-AND-SECOND TRANSMISSION RECEPTION RESULT ADDITION SIGNAL | O | OΔ | O | O |
| EXAMPLE 2 | FIRST-AND-SECOND TRANSMISSION RECEPTION RESULT ADDITION SIGNAL | OΔ | O | O | O |
| EXAMPLE 3 | FIRST-AND-SECOND TRANSMISSION RECEPTION RESULT ADDITION SIGNAL | O | O | OΔ | O |

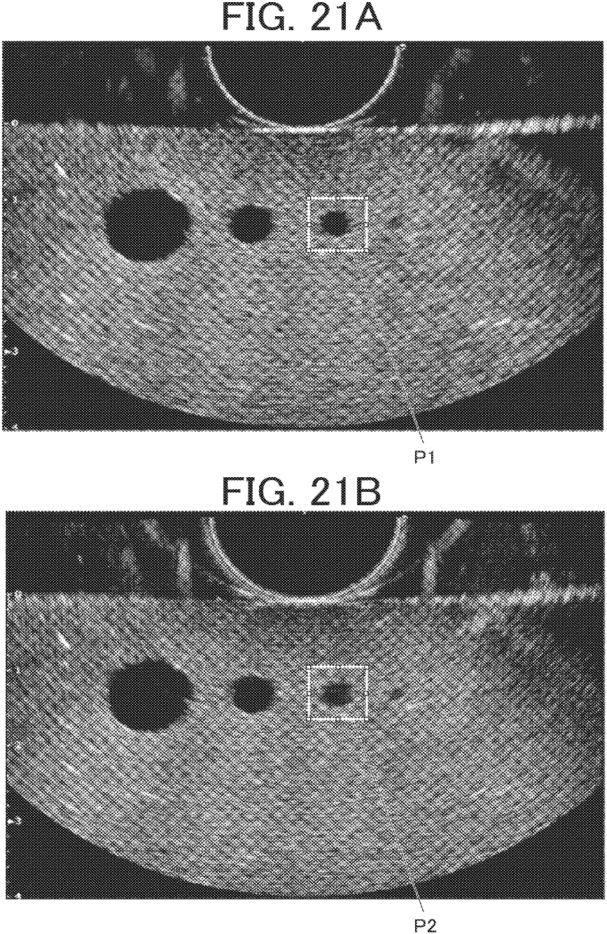

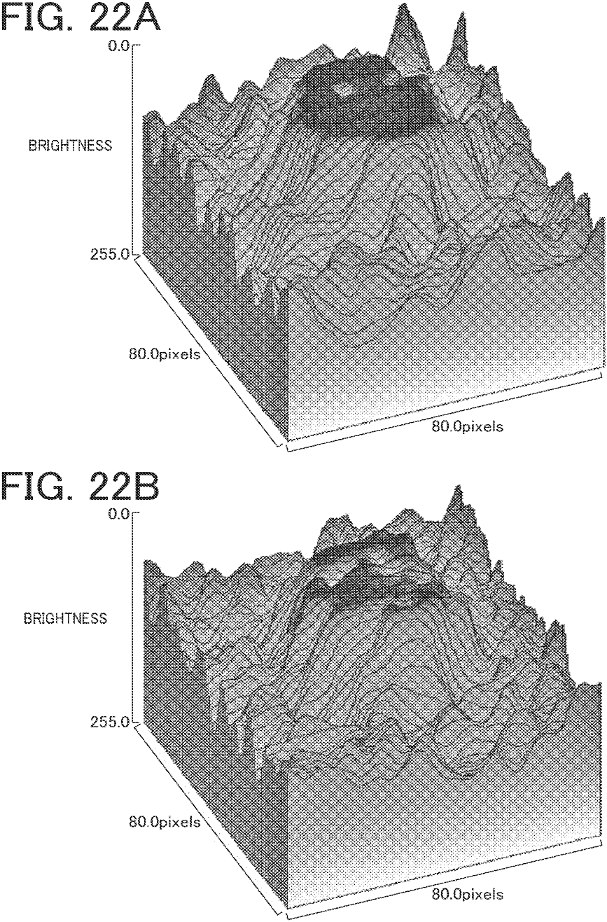

FIG. 21A to FIG. 24B are used to illustrate the image evaluation of the ultrasound image. FIG. 21A shows an ultrasound image of the first subject in Comparative Example 4. FIG. 21B shows an ultrasound image of the first subject in Example 1. FIG. 22A is a 3D graph of the brightness values of the partial area P1 in FIG. 21A.

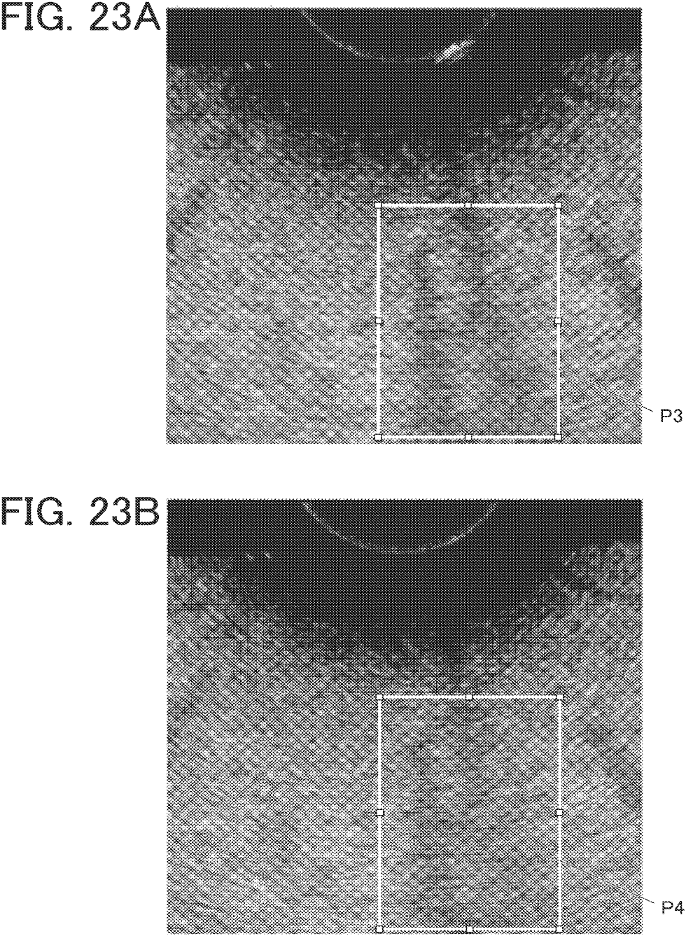

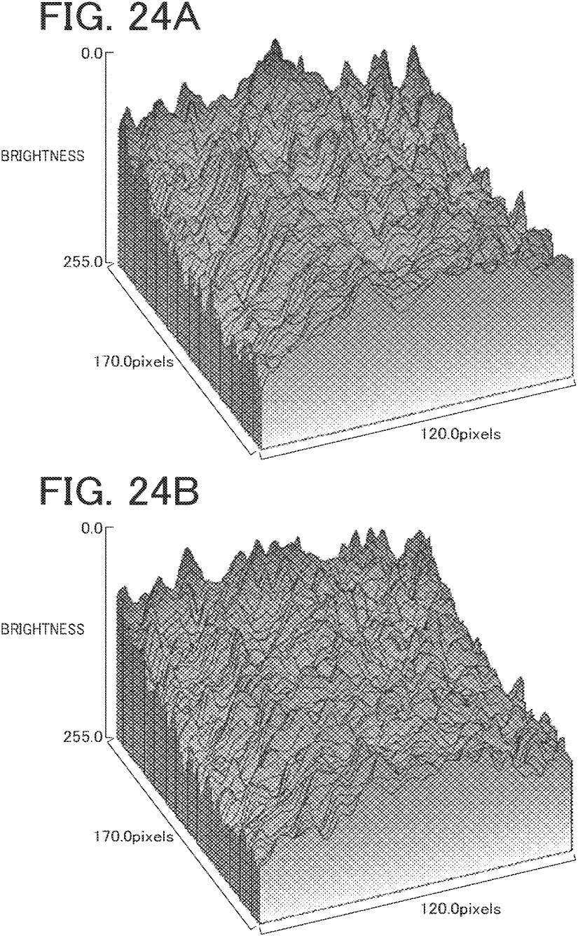

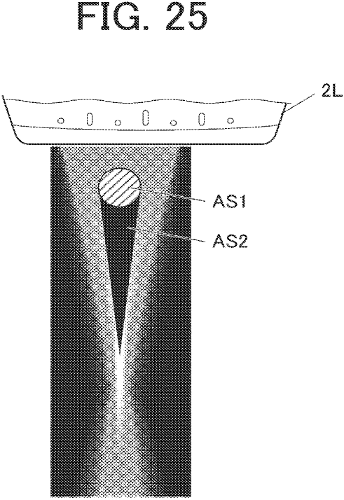

FIG. 22B is a 3D graph of the brightness values of the partial area P2 in FIG. 21B. FIG. 23A shows an ultrasound image of the second subject in Comparative Example 4. FIG. 23B shows an ultrasound image of the second subject in Example 1. FIG. 24A is a 3D graph of the brightness values of the partial area P3 in FIG. 23A. FIG. 24B is a 3D graph of the brightness values of the partial area P4 in FIG. 23B.

First, the ultrasound image data was generated by scanning the same first subject in the ultrasound diagnostic apparatus S, using the transmission conditions and imaging signal conditions of Comparative Examples 1-4 and Examples 1-3. The first subject was a custom-made phantom (Cyst depth 1-2 cm) based on Gammex 404GS as the matrix part (material part of the scatterer) with the addition of a Cyst part (small diameter cyst target (uniform material part with non-echoic component that mimics a cyst)).

For the “Shallow Cyst Delineation” of the image evaluation in Table II, the degree of reduction of the Cyst part size and the degree of change in brightness of the boundary of the Cyst part in the ultrasound image of Comparative Example 4 was set to the cross mark level, the degree of reduction of the Cyst part size and the degree of change in brightness of the boundary of Example 1 was set to the circle level, and the results were assigned based on the results of both items for each example or comparative example by equally dividing from the cross mark level to the circle level.

Cyst delineation was good for Comparative Examples 1 and 2 and Examples 1-3, with poor results for Comparative Examples 3 and 4.

For example, the ultrasound image of the first subject’s ultrasound image data in Comparative Example 4 was obtained as shown in FIG. 21A. In this ultrasound image, the area consisting of 80 × 80 pixel pixels including the Cyst part (black circle in the figure) is designated as partial area P1.

In addition, the ultrasound images of the same first subject’s ultrasound image data in Example 1 was obtained as shown in FIG. 21B. In this ultrasound image, the area consisting of 80 × 80 pixel pixels, including the same Cyst part, is designated as partial area P2.

Here, FIG. 22A shows a 3D graph of the brightness value of each pixel in the partial area P1. The X, Y, and Z axes (none of them shown in the figure) were taken so that the partial area P1 is the XY plane and the brightness value is taken on the Z axis. In such a way, the brightness values were graphed in 3D with the low brightness areas being the Z-axis high values in order to improve shape visibility. Also, as shown in FIG. 22B, the brightness value of each pixel in the partial area P2 was graphed in 3D in the same way as in FIG. 22A.

As shown in the 3D graph of brightness in FIG. 22A, in the partial area P1 of Comparative Example 4 with asymmetric PI drive signal output to transducers 2a in all the transmission aperture, the low brightness area size in the Cyst part is reduced and the brightness change at the boundary is gradual. In contrast, as shown in the 3D graph of brightness in FIG. 22B, in the partial area P2 of Example 1, where the symmetric PI drive signal is output to the first transducer group in the inner side of the transmission aperture and the asymmetric PI drive signal is output to the second transducer group in the outer side, the low brightness area size is maintained, and the brightness change at the boundary is steep and the contrast is clear.

Next, the ultrasound image data was generated by scanning the same second subject in the ultrasound diagnostic apparatus S, using the transmission conditions and imaging signal conditions of Comparative Examples 1-4 and Examples 1-3. As a second subject, there was used a phantom which is made of 500 [µmΦ] acrylic particles dispersed in dissolved agar as matrix part (material part of scatterer) as base material, and is solidified with 1 [mm Φ] nylon wire embedded as shadow source (acoustic shielding that is the source of the shadow part).

The “Shadow Reduction Effect” of the image evaluation in Table II is for the shallow and deep portions of the ultrasound image, and based on the brightness uniformity (shallow and deep portions) of the matrix and shadow parts of the comparative Example 1 as cross mark level, the brightness uniformity (shallow portion) of Example 1 as circle triangle level, and brightness uniformity (deep portion) as circle level, and the results were assigned based on the results of both items for each example or comparative example by equally dividing from the cross mark to the circle level.

As for the shadow reduction effects, good results were obtained for Comparative Examples 3 and 4 and Examples 1-3 in both shallow and deep portions, and poor results were obtained for Comparative Examples 1 and 2 in both shallow and deep portions.

For example, the ultrasound image of the first subject’s the ultrasound image data in Comparative Example 4 was obtained as shown in FIG. 23A. In this ultrasound image, the area consisting of 170 × 120 pixel pixels including the shadow part (black portions in the figure) is designated as partial area P3.

The ultrasound image of the same second subject’s the ultrasound image data in Example 1 was obtained as shown in FIG. 23B. In this ultrasound image, the area consisting of 170 × 120 pixel pixels, including the same shadow part, is designated as partial area P4.

Here, FIG. 24A shows a 3D graph of the brightness value of each pixel in the partial area P3. The X, Y, and Z axes (none of them shown in the figure) were taken so that the partial area P3 is the XY plane and the brightness value is taken on the Z axis. In such a way, the brightness values were graphed in 3D with the low brightness areas being the Z-axis high values in order to improve shape visibility, similarly to the Cyst part. Also, as shown in FIG. 24B, the brightness value of each pixel in the partial area P4 was graphed in 3D in the same way as in FIG. 24A.

As shown in the 3D graph of brightness in FIG. 24B, in the partial area P1 of Comparative Example 4 with asymmetric PI drive signal output to transducers 2a in all the transmission aperture, two striated shadow parts are observed from shallow to deep portions as a clear brightness difference. In contrast, as shown in the 3D graph of brightness in FIG. 24B, in the partial area P2 of Example 1 where the symmetric PI drive signal is output to the first transducer group in the inner side of the transmission aperture and the asymmetric PI drive signal is output to the second transducer group in the outer side, the brightness difference with the surrounding area is greatly reduced from the shallow portion (at the back of the 3D graph), and the brightness is generally uniform in the deep portion (at the front of the 3D graph).

For “Penetration (evaluation)” of image evaluation in Table II, two different frames of the ultrasound image data were acquired without time averaging, and the penetration depth is defined as the depth at which the image correlation can be maintained to be 0.5 or greater. The penetration was evaluated to be circle if it was generally equivalent to Example 1, triangle if it was generally equivalent (inferior to Example 1) to Comparative Example 1, and cross mark if it was inferior to Comparative Example 1. However, there were no cross mark evaluations as a result.

For Examples 1-3, all of the image evaluations were good, at the levels (circle triangle or greater) with no practical problems in terms of shallow Cyst delineation, shadow reduction effects, and penetration. By designing the offset residual component to be conditions (d) and (e) in Examples 1-3, a high resolution image delineation has been obtained. In contrast, in Comparative Example 3, in which the reception signal obtained from the transmission ultrasound is directly imaged and the shadow part is covered, the shadow part is covered, but is strongly affected by acoustic noise, and the image signal from the second transmission reception result with a low frequency and narrow band is delineated with a weighting of 50%. The image grain size is coarse and components with poor resolution adversely affect the entire image, and thus, for example, Cyst delineation (shallow Cyst delineation) is degraded.

Comparative Example 4, which used the first and second drive signals with offset residual without dividing the transmission aperture into the first transducer group in the inner side and the second transducer group in the outer side, was strongly affected by acoustic noise due to the offset residual component from the shallow portion, resulting in poorer shallow Cyst delineation compared to Examples 1 to 3. In addition, in Example 1 and Example 2 which has fewer elements in the first transducer group in the inner side than Example 1, setting the same the transmission aperture, the shallow Cyst delineation was better for Example 1 than Example 2, because Example 1 is less affected by the offset residual component of the second transducer group in the outer side. For the shadow reduction effect (shallow portion), Example 2 was better than Example 1 because the transmission ultrasound from the second transducer group, which is on the outer side, is more likely to go around the shadow part. Example 3, in which both the number of elements of the first vibrating element group (inner) and the number of elements of the second vibrating element group (outer) were the same, and the intensity of the offset residual component was changed by changing the drive waveform of the second vibrating element group (outer), improved shallow Cyst delineation, but the shadow reduction effect (deep portion) was better for Example 2 than Example 3 because the ratio of the offset residual component to the total imaging signal was reduced.

As described above, according to the embodiment, an ultrasound diagnostic apparatus S generates the ultrasound image data on the basis of the reception signal obtained by the ultrasound probe 2 that transmits the transmission ultrasound to a subject and receives the reception ultrasound from the subject. The ultrasound probe 2 includes the transmission aperture including a plurality of transducers 2. The transmission aperture includes a plurality of transducer groups including at least a first transducer group arranged in the inner side of the transmission aperture and a second transducer group arranged in the outer side than the first transducer group. The ultrasound diagnostic apparatus S includes: a transmitter 12 that generates a drive signal and outputs the drive signal to the plurality of transducer groups; a controller 18 that causes the transmitter 12 to output a plurality of drive signals including at least a first drive signal and a second drive signal to the plurality of transducer groups for one focusing line; a receiver 13 that receives a plurality of reception signals corresponding to the plurality of drive signals from the ultrasound probe 2; and an image generator 14 that calculates the plurality of reception signals and generates ultrasound image data. The first drive signal includes a third drive signal and a fourth drive signal. The second drive signal includes a fifth drive signal that is offset when added to the third drive signal and a sixth drive signal that generates an offset residual when added to the fourth drive signal. The controller 18 causes the transmitter 12 to provide a time delay to the first drive signal and the second drive signal so that the transmission ultrasound focuses to a same focal point, output the third drive signal and the fifth drive signal to the first transducer group arranged in the inner side, and output the fourth drive signal and the sixth drive signal to the second transducer group arranged in the outer side.

Therefore, in the presence of acoustic shields, the reduction in dark portion delineation due to acoustic noise can be eliminated, and good ultrasound images with shadow tolerance can be obtained. The asymmetric PI third and fifth drive signals can improve penetration while maintaining resolution.

The image generator 14 generates the ultrasound image data by adding the reception signals. Therefore, the higher harmonic wave component provides ultrasound images with high resolution, low artifacts, and good contrast.

The first drive signal and the second drive signal have a same drive voltage. This prevents the complex configuration of separately setting drive voltages for the first and second drive signals, the first transducer group in the inner side, and the second transducer group in the outer side, and simplifies the configuration of the ultrasound diagnostic apparatus S and reduces costs.

The central frequency of a frequency power spectrum (asymmetric PI signal component OTx) of transmission ultrasound of the fourth drive signal and the sixth drive signal is smaller than a central frequency of a frequency power spectrum (symmetric PI signal component ITx) of transmission ultrasound of the third drive signal and the fifth drive signal. The bandwidth of a frequency power spectrum (asymmetric PI signal component OTx) of transmission ultrasound of the fourth drive signal and the sixth drive signal is smaller than a bandwidth of a frequency power spectrum (symmetric PI signal component ITx) of transmission ultrasound of the third drive signal and the fifth drive signal. In a higher frequency area than a central frequency of the ultrasound probe 2, a signal intensity of a frequency power spectrum (asymmetric PI signal component OTx) of transmission ultrasound of the fourth drive signal and the sixth drive signal is smaller than a signal intensity of a frequency power spectrum (symmetric PI signal component ITx) of transmission ultrasound of the third drive signal and the fifth drive signal. This can improve image delineation of no-to-low-echo areas of shallow portion.

The central frequency of a frequency power spectrum of transmission ultrasound of the third drive signal and the sixth drive signal is smaller than a central frequency of a component of the offset residual. The bandwidth of a frequency power spectrum of transmission ultrasound of the third drive signal and the sixth drive signal is smaller than a bandwidth of the offset residual. Therefore, for the shadow part, the wide bandwidth allows for obtaining an image delineation with fine image granularity and high resolution.

According to the embodiments, good ultrasound images can be obtained in the presence of the acoustic shield.

The description in the above embodiment is an example of a preferred ultrasound diagnostic apparatus according to the present invention and the present invention is not limited thereto.

For example, in the above embodiment, the transducers 2a of the transmission aperture of the ultrasound probe 2 are divided into a first transducer group, which is tin the inner side, and a second transducer group, which is in the outer side, but this is not a limitation. The transducers 2a of the transmission aperture of the ultrasound probe 2 may be divided into three or more transducer groups, including a first transducer group in the inner side and a second transducer group in the outer side. In the above embodiment, one transducer group is described as having a configuration with plurality of transducers 2a, but it is not limited to this configuration and one transducer group may have at least one transducer 2a.

As for the detailed configurations and the detailed operation of the components forming the ultrasound diagnostic apparatus S in the above embodiment, modifications can be made as needed within the scope of the present invention.

Although embodiments of the present invention have been described and illustrated in detail, the disclosed embodiments are made for purposes of illustration and example only and not limitation. The scope of the present invention should be interpreted by terms of the appended claims.

1. An ultrasound diagnostic apparatus that generates ultrasound image data based on a reception signal obtained by an ultrasound probe that transmits transmission ultrasound to a subject and receives reception ultrasound from the subject, wherein the ultrasound probe includes a transmission aperture including a plurality of transducers, and the transmission aperture includes a plurality of transducer groups including at least a first transducer group that is arranged in an inner side of the transmission aperture and a second transducer group that is arranged in an outer side than the first transducer group, the ultrasound diagnostic apparatus comprising:
a transmitter that generates a drive signal and outputs the drive signal to the plurality of transducer groups;
a hardware processor that causes the transmitter to output a plurality of drive signals including at least a first drive signal and a second drive signal to the plurality of transducer groups for one focusing line;
a receiver that receives a plurality of reception signals corresponding to the plurality of drive signals from the ultrasound probe; and
an image generator that calculates the plurality of reception signals and generates ultrasound image data, wherein
the first drive signal includes a third drive signal and a fourth drive signal,
the second drive signal includes a fifth drive signal that is offset when the fifth drive signal and the third drive signal are added, and a sixth drive signal that generates an offset residual when the sixth drive signal and the fourth drive signal are added, and
the hardware processor causes the transmitter to provide a time delay to the first drive signal and the second drive signal so that the transmission ultrasound focuses to a same focal point, output the third drive signal and the fifth drive signal to the first transducer group, and output the fourth drive signal and the sixth drive signal to the second transducer group.

2. The ultrasound diagnostic apparatus according to claim 1, wherein the image generator generates the ultrasound image data by adding the reception signals.

3. The ultrasound diagnostic apparatus according to claim 1, wherein the first drive signal and the second drive signal have a same drive voltage.

4. The ultrasound diagnostic apparatus according to claim 1, wherein a central frequency of a frequency power spectrum of transmission ultrasound of the fourth drive signal and the sixth drive signal is smaller than a central frequency of a frequency power spectrum of transmission ultrasound of the third drive signal and the fifth drive signal.

5. The ultrasound diagnostic apparatus according to claim 1, wherein a bandwidth of a frequency power spectrum of transmission ultrasound of the fourth drive signal and the sixth drive signal is smaller than a bandwidth of a frequency power spectrum of transmission ultrasound of the third drive signal and the fifth drive signal.

6. The ultrasound diagnostic apparatus according to claim 1, wherein, in a higher frequency area than a central frequency of the ultrasound probe, a signal intensity of a frequency power spectrum of transmission ultrasound of the fourth drive signal and the sixth drive signal is smaller than a signal intensity of a frequency power spectrum of transmission ultrasound of the third drive signal and the fifth drive signal.

7. The ultrasound diagnostic apparatus according to claim 1, wherein a central frequency of a frequency power spectrum of transmission ultrasound of the third drive signal and the sixth drive signal is smaller than a central frequency of a component of the offset residual.

8. The ultrasound diagnostic apparatus according to claim 1, wherein a bandwidth of a frequency power spectrum of transmission ultrasound of the third drive signal and the sixth drive signal is smaller than a bandwidth of a component of the offset residual.
